(19) United States
(12) Patent Application Publication
(10) Pub. No.: US 2026/0369371 A1
(43) Pub. Date: Nov. 19, 2026

(54) GENETIC HEALTHCARE GUIDANCE SYSTEM

(71) Applicant: INTELLIGENE SA (PTY) LTD, Pretoria (ZA)

(72) Inventors: Heinrich Carl Wilhelm RIX, Pretoria (ZA); McElory Roberto HOFFMANN, Pretoria (ZA); Mathilde Elizabeth VERSTER, Pretoria (ZA)

(73) Assignee: INTELLIGENE SA (PTY) LTD, Pretoria (ZA)

(21) Appl. No.: 19/752,352

(22) Filed: Jul. 23, 2026

Related U.S. Application Data
(63) Continuation-in-part of application No. 18/870,945, filed on May 31, 2023, now abandoned.

(30) Foreign Application Priority Data
Jun. 2, 2022 (ZA) 2022/06137

(51) Int. Cl. G16H 10/60; G16H 70/20

(52) U.S. Cl. CPC G16H 10/60; G16H 70/20

(57) ABSTRACT

A genetic healthcare guidance system which includes: a processor with access to a memory containing instructions executable by the processor to perform operations including: a data acquisition operation for acquiring individual-specific user data from an individual, the individual-specific user data including user genetic information, user medication information, and user health condition information; and a multi-phase computational mapping operation for mapping the individual-specific user data to healthcare guidance data contained in a mapping database to generate an output including mapped gene-specific healthcare guidance information associated with the individual, the multi-phase computational mapping operation including: a gene-mapping phase for mapping the user genetic information to gene-related data contained in the mapping database to generate mapped gene-related data associated with the individual; a medication-mapping phase for mapping the user medication information to medication-related data contained in the mapping database to generate mapped medication-related data associated with the individual; and a healthcare-mapping phase for mapping the mapped gene-related data and the mapped medication-related data to healthcare-related data contained in the mapping database to generate mapped healthcare data associated with the individual to be included in the output.

### CROSS-REFERENCE TO RELATED APPLICATIONS

This application is a continuation-in-part of U.S. application Ser. No. 18/870,945 filed Dec. 2, 2024, which is a national stage application of PCT Application No. PCT/ZA2023/050029 filed May 31, 2023, which claims priority to South Affrica Application No. 2022/06137 filed Jun. 2, 2022, which applications are incoporated herein by specific reference in their entirety.

### TECHNICAL FIELD

This invention relates to a genetic healthcare guidance system. In particular, this invention relates to a genetic healthcare guidance system for generating gene-specific healthcare guidance information associated with an individual.

### SUMMARY OF THE INVENTION

According to a first aspect of the invention, there is provided a genetic healthcare guidance system which includes:
a processor with access to a memory containing instructions executable by the processor to perform operations including:
a data acquisition operation for acquiring individual-specific user data from an individual, the individual-specific user data including user genetic information, user medication information, and user health condition information; and
a multi-phase computational mapping operation for mapping the individual-specific user data to healthcare guidance data contained in a mapping database to generate an output including mapped gene-specific healthcare guidance information associated with the individual, the multi-phase computational mapping operation including:
a gene-mapping phase for mapping the user genetic information to gene-related data contained in the mapping database to generate mapped gene-related data associated with the individual;
a medication-mapping phase for mapping the user medication information to medication-related data contained in the mapping database to generate mapped medication-related data associated with the individual;
a healthcare-mapping phase for mapping the mapped gene-related data and the mapped medication-related data to healthcare-related data contained in the mapping database to generate mapped healthcare data associated with the individual to be included in the output.

A display device may be provided for receiving and displaying visual output data generated by the processor. The display device may be accessible and controllable by the processor. The display device may include any suitable hardware configured to display the visual output data. The suitable hardware may include any of the group including a computer monitor, a mobile device screen, and a tablet display.

The processor may include any suitable hardware device which is configured to execute the instructions. The hardware device may include any one or more of the group including a processing unit, micro-processor, and microcontroller. The hardware device may form part of a plurality of interconnected processing units. The plurality of interconnected processing units may form a distributed computing network or a cloud-based server architecture. The processor may be configured to output a user interface to the display device. The user interface may be configured to facilitate user interaction with the system. The user interface may include any one or more of the group including a graphical user interface (GUI), a web-based portal, and a dedicated application interface.

The memory may include a non-transitory computer-readable storage medium within which the instructions are stored. The memory may be arranged in communication with the processor to enable execution of the instructions by the processor. The storage medium may be further configured to host one or more databases or repositories for securely storing system data.

The instructions may be in the form of computer-readable instructions for performing the operations. In particular, the instructions may be in the form of executable computer code. The computer code may be logically structured as a plurality of distinct software modules or integrated into a unified computational framework to execute the operations.

The system may include a profile creation operation for creating profiles for users of the system and storing the profiles on a repository. The profile creation operation may be configured to create an individual profile for a user in the form of an individual. The profile creation operation may be configured to create a practitioner profile for a user in the form of a health practitioner.

The profile creation operation may include a credential acquisition operation for acquiring credentials from the user. The credential acquisition operation may be initiated upon user selection of a selectable profile creation element.

The credential acquisition operation may include displaying a credential acquisition visual to the user via the user interface. The credential acquisition visual may include a plurality of credential capture elements configured to receive the credentials from the user. The plurality of credential capture elements may include any one or more of the group including a manual text entry field, a file upload interface, and an external database integration module. The file upload interface may be configured to receive electronic documents containing the credentials. The external database integration module may be configured to establish a communication link with a remote credential database to automatically extract the credentials.

The credentials may include login details. The login details may include a unique identifier and an authenticator. The unique identifier may be in the form of any of the group including a name, username, full name, identification number, email address, telephone number, cellular number. The authenticator may be in the form of any of the group including a password, code, OTP (one-time-pin), personal identification number (PIN), passcode, time-based one-time password (TOTP), biometric input, fingerprint scan, facial recognition data, iris scan, voiceprint, hardware security token, software security token, cryptographic key, authentication link, spatial pattern, and security question response.

The credentials may include user type information. The user type information may include a user type identifier. The user type identifier may be an identifier of a user type. The user type may include individual type and health practitioner type. Input of the user type identifier serves to enable the system to differentiate between individuals and health practitioners.

The profile creation operation may include an allocation operation for allocating a dedicated memory space within the repository to the user. The profile creation operation may include a credential storing operation for storing the credentials within the dedicated memory space. The profile creation operation may include a profile identifier generation operation for generating a unique profile identifier and assigning the unique profile identifier to the dedicated memory space. The allocation operation, credential storing operation and profile identifier generation operation may be initiated upon user selection of a selectable confirmation element displayed to the user within the credential acquisition visual. It is to be appreciated that the profiles may be defined by the unique profile identifiers and contents of the memory space dedicated to the users. The profiles may be configured to represent distinct digital data records for the individuals and health practitioners, respectively.

The profile creation operation may be configured to establish registered users, wherein the registered users include individuals and health practitioners having dedicated profiles stored on the repository.

It is to be appreciated that generating a distinct health practitioner profile provides the technical advantage of enabling role-based access control and secure data segregation within the system. In particular, utilising a separate health practitioner profile allows the system to execute isolated authentication processes and manage targeted access permissioning independently of the individual profile, thereby reducing data duplication and enhancing the secure handling of sensitive clinical information.

The repository may be accessible by the processor. The repository may form part of a remote user database. The remote user database may be in the form of a cloud-based database.

The individual may include any human subject. The human subject may include a primary user of the system seeking healthcare guidance. The primary user may include any one or more of the group including a patient, a prospective patient, a consumer, and a research participant. The individual may include a representative acting on behalf of the primary user. The representative may include any one or more of the group including a legal guardian, a parent, a caregiver, and a designated proxy. The designated proxy may be authorised to create and manage the individual profile on behalf of a dependent. The dependent may include a minor child or a legally incapacitated adult.

The health practitioner may be in the form of any person engaged in human health as a profession. In particular the health practitioner may be in the form of any one or more of the group including, but not limited to, a medical doctor, physician, dietician, optometrist, ophthalmologist, pharmacist, physical therapist, occupational therapist, dentist, psychologist, psychiatrist, sports trainer, genetic specialist, pathologist, nurse practitioner, midwife, chiropractor, speech therapist, audiologist, radiologist, anaesthesiologist, dermatologist, neurologist, cardiologist, pulmonologist, gastroenterologist, endocrinologist, urologist, orthopaedic surgeon, oncologist, rheumatologist, geriatrician, paediatrician, nutritionist, immunologist, allergist, naturopath, homeopath, acupuncturist, and reflexologist.

The data acquisition operation may include storing the individual-specific user data against the individual profile. The data acquisition operation may include displaying a user data acquisition visual to the user via the user interface. The user data acquisition visual may include a plurality of data capture elements configured to receive the individual-specific user data. The plurality of data capture elements may include any one or more of the group including a manual text entry field, a file upload interface, and an external database integration module. The file upload interface may be configured to receive electronic documents containing the individual-specific user data. The external database integration module may be configured to establish a communication link with a remote user information database to automatically extract the individual-specific user data.

The data acquisition operation may further include a dynamic data updating operation. The dynamic data updating operation may be executed asynchronously to continuously maintain the individual-specific user data. The dynamic data updating operation may be configured to permit manual updating of the individual-specific user data by an authorised third party. The authorised third party may include an authorised health practitioner or an authorised medical aid service provider. The manual updating may include updating the user medication information and the user health condition information 28 to reflect current drug prescriptions, identified symptoms, medical test results, or the presence of illness subsequent to a clinical consultation.

The dynamic data updating operation may be configured to permit automatic updating of the individual-specific user data. The automatic updating may be executed by the external database integration module. The automatic updating may include automatically receiving continuous health-related data from a network-connected smart device associated with the individual. The automatic updating may further include securely querying an external database associated with the medical aid service provider to automatically extract updated user medication information and/or user health condition information. It is to be appreciated that executing the dynamic data updating operation ensures the system continuously reflects the current health status of the individual, thereby computationally triggering dynamic re-mapping operations in response to newly acquired user medication information and/or user health condition information.

The individual-specific user data may include user identification information. The user identification information may include any one or more of the group including identification number, name, surname, full names, username, and residential address. The identification information may include contact information. The contact information may include any one or more of the group including email address, mobile number, and contact details of third parties. The third parties may include any of the group including family members, legal guardians, and friends.

The individual-specific user data may include the user genetic information. The user genetic information may include foundational genotypic data of the individual. The foundational genotypic data may include uninterpreted biological data. The uninterpreted biological data may include any one or more of the group including genetic variants, single nucleotide polymorphisms (SNPs), specific genetic alleles, gene expression profiles, chromosomal abnormalities, DNA methylation patterns, epigenetic modifications, copy number variations, mitochondrial DNA data, haplogroups, structural variations, non-coding RNA analysis, carrier status for inherited conditions, and ancestry or ethnic origins. The user genetic information may further include genetic testing results. The genetic testing results may include raw genotyping data and raw sequencing data. The user genetic information may include a digital genetic file. The digital genetic file may include an output file generated by a third-party genetic testing laboratory. The user genetic information may further include historical user inputs. The historical user inputs may include familial genetic history and genetic counselling records. It is to be appreciated that the user genetic information may be obtained via a processed DNA sample of the user. The DNA sample may typically be in the form of a buccal swab or a blood sample, preferably being in the form of a buccal swab. It is to be appreciated further that the processed DNA sample typically produces genetic data capable of being stored on the repository against the individual profile.

The individual-specific user data may include the user medication information. The user medication information may include historical and/or current user medication information. The user medication information may include information relating to any one or more of the group including medications, prescribed drugs, dietary supplements, herbal remedies, and alternative medicine usage.

The individual-specific user data may include the user health condition information. The user health condition information may include historical and/or current user health condition data. The user health condition information may include any health-related information which may influence or relate to a health condition of a human being. The health-related information may include any one or more of the group including medical concerns, illnesses, diagnoses, treatments, previous medical practitioners, family medical history, sleep quality, stress levels, exercise regimes, allergies, known adverse drug reactions, vaccination history, surgical procedures, wound healing progress, surgical recovery status, lifestyle habits, dietary information, mental health history, cognitive function, reproductive health, pregnancy or lactation status, laboratory test results, renal function indicators, hepatic function indicators, imaging reports, vital signs (including blood pressure, blood oxygen levels, heart rate, body temperature, and respiratory rate), biometric data (including weight, height, and body mass index), blood type, immunisations, social determinants of health, substance and alcohol use history, environmental exposures, preventive screenings, therapeutic interventions, rehabilitation progress, chronic conditions, physical disabilities, implanted or wearable medical devices, patient-reported outcomes, and organ donation preferences. The user health condition information may further include occupational data relating to the nature of the user's employment, preferably including a measure of a degree of stress experienced by users in a particular employment type.

The individual-specific user data may include user activity data. The user activity data may include historical and/or current user activity data. The user activity data may include any one or more of the group including a historical record of overall measurement of activity and/or inactivity, steps, heart rate, exercises, training sessions, specific sports associated with the user, duration of physical activities, calories burned, distance covered, average pace or speed, workout intensity levels, rest periods, sleep patterns, sedentary time, active time, exercise goals and achievements, training plans, fitness assessments, heart rate variability, recovery metrics, exercise equipment usage, workout classes attended, personal bests, performance metrics, exercise-related injuries, exercise frequency, exercise preferences, and training load.

The individual-specific user data may include user medical aid data. The user medical aid data may include historical and/or current user medical aid data. The user medical aid data may include any of the group including medical aid name, medical aid type, medical aid number, medical aid provider, medical aid scheme name, plan option, benefit tier, policy number, primary member identifier, dependent code, beneficiary number, principal member details, coverage limits, benefit exclusions, available medical savings balance, co-payment requirements, excess parameters, claims history, pre-authorisation records, coverage inception date, coverage expiration date, and funding availability.

While the individual-specific user data may include any combination of the aforementioned data categories, execution of the multi-phase mapping operation specifically relies upon the acquisition of at least the user genetic information and the user medication information. Accordingly, the individual-specific user data may includes at least the user genetic information and the user medication information.

The data acquisition operation may include a data storing operation for storing acquired individual-specific user data against the individual profile. The data storing operation may be initiated upon receipt of the individual-specific user data via the data capture elements. Storing the individual-specific user data against the individual profile may include writing the individual-specific user data into the dedicated memory space allocated to the individual. The data storing operation may include associating the acquired individual-specific user data with the unique profile identifier assigned to the dedicated memory space. It is to be appreciated that associating the acquired individual-specific user data with the unique profile identifier ensures structural database integrity and enables the processor to accurately retrieve the individual-specific user data during the multi-phase mapping operation.

The data acquisition operation may be executed sequentially and immediately following the profile creation operation within a continuous individual onboarding session. In such an embodiment, the processor may be configured to display the data acquisition visual automatically upon creation of the individual profile. The data acquisition operation may be executed asynchronously at any time subsequent to the profile creation operation upon successful authentication of the individual using the login details.

The system may include a practitioner verification operation for verifying a professional status of the health practitioner. The practitioner verification operation may be initiated by the processor upon determining that the user type identifier corresponds to the health practitioner.

The practitioner verification operation may include a practitioner data acquisition operation. The practitioner data acquisition operation may include displaying a verification visual to the health practitioner via the user interface. The verification visual may include a plurality of data capture elements configured to receive practitioner-specific user data.

The practitioner-specific user data may include any one or more of the group including field of practice, associated governing bodies, professional credentials, license identifier, practice identifier, regulatory registration details, national healthcare provider identifier, medical board license number, prescribing identifier, controlled substance registration number, professional council registration number, board certification identifier, specialisation classification code, national or regional registry number, professional association membership number, health facility identification number, and digital practitioner certificate.

The practitioner data acquisition operation may be executed sequentially and immediately following the profile creation operation within a continuous practitioner onboarding session. In such an embodiment, the processor may be configured to display the verification visual automatically upon creation of the practitioner profile. The practitioner data acquisition operation may be executed asynchronously at any time subsequent to the profile creation operation upon successful authentication of the health practitioner using the login details.

The practitioner verification operation may further include a practitioner data validation operation. The practitioner data validation operation may include computationally comparing the received practitioner-specific user data against a verification database. The verification database may include a remote regulatory database accessible by the processor. The computational comparison may include transmitting an automated query to the remote regulatory database, the query containing at least a portion of the practitioner-specific user data.

The practitioner data validation operation may further include receiving a validation response from the remote regulatory database. The validation response may include a verification confirmation or a verification denial. The practitioner data validation operation may include updating a verification status of the health practitioner profile based on the received validation response. Updating the verification status may include assigning a verified state to the dedicated memory space associated with the health practitioner upon receipt of the verification confirmation.

The system may include a use prohibition operation for prohibiting performance of the authorisation operation if the professional status of the health practitioner is not verified. The use prohibition operation may be initiated by the processor upon receipt of the verification denial or upon determining that the dedicated memory space associated with the health practitioner lacks the verified state. Prohibiting performance of the authorisation operation may include technical enforcement by the processor. On a user interface level, the technical enforcement may include disabling or hiding one or more selectable elements associated with the authorisation operation on the display device. On a database level, the technical enforcement may include the processor restricting read and write access to the dedicated memory space defining the individual profile. It is to be appreciated that ensuring that only verified medical professionals may use the system prevents unverified practitioners or unauthorised nodes from being granted access to sensitive and/or confidential information stored against individual profiles, thereby maintaining cryptographic and structural data security.

The mapping database may be accessible by the processor.

The mapping database may include any one or more data groups including phenotypic data, pharmacogenetic data, drug-drug interaction data, pharmacogenomic data, biological data, pharmacological data, and healthcare guidance data.

The phenotypic data may include a plurality of observable characteristics structurally linked to specific gene variations. The plurality of observable characteristics may include metabolic phenotypes. The metabolic phenotypes may include metaboliser status categories which may relate to metabolism rates of active pharmaceutical ingredients. The metaboliser status categories may be selected from any one or more of the group including ultra-rapid metabolisers, extensive metabolisers, intermediate metabolisers, and poor metabolisers.

The pharmacogenetic data may include a plurality of drug reactions structurally linked to specific gene variations. The plurality of drug reactions may include specific pharmacological responses to active pharmaceutical ingredients.

The drug-drug interaction data may include a plurality of known drug-drug contraindications structurally linked to specific active pharmaceutical ingredients. The drug-drug interaction data may include metabolic pathway interference profiles. The metabolic pathway interference profiles may define pharmacokinetic interactions between the plurality of active pharmaceutical ingredients. The drug-drug interaction data may include severity classifications. The severity classifications may be indicative of a clinical risk level associated with the known contraindications. It is to be appreciated that the drug-drug interaction data provides a generalised computational reference for identifying adverse polypharmacy effects independent of genetic factors.

The mapping database may further include pharmacogenomic data. The pharmacogenomic data may include a plurality of likely drug reactions structurally linked to active pharmaceutical ingredients and specific gene variations. In particular, the pharmacogenomic data may include a plurality of biological pathways structurally linked to active pharmaceutical ingredients and specific gene variations. The plurality of biological pathways may include pharmacokinetic and pharmacodynamic pathways. The pharmacogenomic data may further include therapeutic classifications structurally linked to the active pharmaceutical ingredients. The therapeutic classifications may define categorical groupings of the active pharmaceutical ingredients based on shared clinical purposes or common mechanisms of action. It is to be appreciated that the therapeutic classifications provide a structured computational reference enabling the system to extract suitable alternative therapies from within a shared categorical grouping when a specific active pharmaceutical ingredient is contraindicated. It is to be appreciated that the pharmacogenomic data provides a relational data model for grouping the active pharmaceutical ingredients by shared biological pathways and therapeutic classes.

The mapping database may include biological data. The biological data may include any one or more of the group including disease-associated genetic markers, genotype-phenotype correlations, non-coding RNA analysis, gene function annotations, protein-protein interaction networks, pathway analysis results, regulatory elements, disease risk scores, polygenic risk scores, genetic traits or phenotypes, gene ontology annotations, genetic correlations, genome-wide association study (GWAS) results, and population-level genetic data.

The mapping database may include pharmacological data. The pharmacological data may include uses, effects and modes of action structurally linked to specific drugs. The pharmacological data may include any one or more of the group including established drug-gene interaction pathways, adverse event probabilities, therapeutic efficacy thresholds, drug-herb interaction data, and toxicity risk factors.

The healthcare guidance data may include clinical guidance data. The clinical guidance data may include a plurality of actionable clinical recommendations. The plurality of actionable clinical recommendations may be structurally linked to the phenotypic data, pharmacogenetic data and the drug-drug interaction data. Preferably, the plurality of actionable clinical recommendations may be structurally linked to the phenotypic data, pharmacogenetic data, the drug-drug interaction data, the pharmacogenomic data, the biological data, and the pharmacological data. The plurality of actionable clinical recommendations may be selected from any of the group including dosing recommendations, alternative treatment suggestions, risk stratification classifications, clinical monitoring protocols, cautionary clinical flags, and structured clinical action mappings. The structured clinical action mappings may dictate specific actions the health practitioner should take in response to any one or more of the group including a specific metabolic phenotype, a specific gene-drug interaction, a specific drug-drug interaction, a specific biological pathway taken by a specific pharmaceutical active ingredient, a specific biological data indication, and a specific pharmacological data indication. The alternative treatment suggestions may be selected from any of the group including an alternative medication, an alternative therapeutic class, and an adjusted dosage regimen. The risk stratification classifications may be selected from any of the group including toxicity risks, efficacy predictors, and metabolism rates.

The healthcare guidance data may include age-specific rules. The age-specific rules may be selected from any of the group including standard adult-based guidelines and paediatric-specific recommendations.

The healthcare guidance data may include curated scientific source data. The curated scientific source data may be selected from any of the group including pharmacogenomic consortia guidelines, regulatory labelling, and peer-reviewed literature. The curated scientific source data may be configured to provide traceable reference links. It is to be appreciated that the traceable reference links ensure that the genetic-specific healthcare guidance is clinically validated and transparently verifiable by the health practitioner.

The mapping database may be configured as a relational database architecture. The data groups may be structurally linked via a plurality of associative data tables. The structural linking may include assigning a unique primary key to discrete data elements within each of the data groups. The structural linking may further include embedding a plurality of foreign keys across the associative data tables to computationally bind the data groups together.

The data groups may be computationally categorised into database domains. The database domains may overlap with each other. The database domains may include gene-related data, medication-related data, and healthcare-related data. The gene-related data may include the phenotypic data, the biological data, the pharmacogenetic data, and the pharmacogenomic data. The medication-related data may include the pharmacological data, the drug-drug interaction data, the pharmacogenetic data, and the pharmacogenomic data. The healthcare-related data may include the healthcare guidance data. It is to be appreciated that assigning specific data groups, such as the pharmacogenetic data and the pharmacogenomic data, to both the gene-related data and the medication-related data creates a computational overlap. The computational overlap establishes a relational bridge within the mapping database. The relational bridge enables the processor to execute the multi-phase computational mapping operation, allowing a relational query to automatically traverse from the mapped gene-related data, through the overlapping data groups, to extract the corresponding mapped medication-related data and the mapped healthcare-related data without requiring manual human interpretation.

In an alternative form, the mapping database may be configured as a directed graph database architecture. The data groups may be structurally linked via a plurality of computational nodes and directed edges. The structural linking may include storing discrete data elements within each of the data groups as discrete computational nodes. The structural linking may further include computationally binding specific computational nodes together using the directed edges. Each directed edge may include a machine-readable relationship property, such as a biological pathway dependency, a metabolic rate interaction, or a drug-drug contraindication.

The data groups may be computationally categorised into database domains. The database domains may overlap with each other. The database domains may include gene-related data, medication-related data, and healthcare-related data. The gene-related data may include the phenotypic data, the biological data, the pharmacogenetic data, and the pharmacogenomic data. The medication-related data may include the pharmacological data, the drug-drug interaction data, the pharmacogenetic data, and the pharmacogenomic data. The healthcare-related data may include the healthcare guidance data. It is to be appreciated that assigning specific data groups, such as the pharmacogenetic data and the pharmacogenomic data, to both the gene-related data and the medication-related data creates a computational overlap. In the directed graph database architecture, the computational overlap is established by shared computational nodes and interconnecting directed edges. This computational overlap establishes a continuous associative path within the mapping database. The continuous associative path enables the processor to execute the multi-phase computational mapping operation, allowing a graph traversal to automatically trace from the mapped gene-related data, along the directed edges through the overlapping data groups, to extract the corresponding mapped medication-related data and the mapped healthcare-related data without requiring manual human interpretation.

The mapping database may be in the form of a computational knowledge base. It is to be appreciated that configuring the mapping database as a computational knowledge base ensures that the contents are structurally embedded with actionable logic for automated inference rather than passive data retrieval, thereby functioning as a structural component of a processing engine. The computational knowledge base may include a plurality of machine-readable logical relationships. The machine-readable logical relationships may structurally define interactions between the data groups of the mapping database. The machine-readable logical relationships may be executed via the foreign keys of the relational database architecture or the directed edges of the directed graph database architecture.

The computational knowledge base may include computable clinical logic. The computable clinical logic may provide machine-readable instructions which are executable to computationally govern the multi-phase mapping operation. It is to be appreciated that the computable clinical logic translates qualitative clinical guidelines into deterministic, machine-readable instructions, thereby enabling the processor to automatically execute the multi-phase mapping operation without requiring human clinical interpretation.

The machine-readable instructions may include conditional algorithms. The conditional algorithms may be configured to computationally govern dynamic evaluation of state-dependent variables. The state-dependent variables may include dynamic clinical parameters derived from the user health condition information. It is to be appreciated that while the user genetic information serves as a static biological baseline for the individual, the dynamic clinical parameters represent an ever-changing clinical snapshot, wherein a modification to the user health condition information alters a computational state of the system and necessitates algorithmic re-execution of the computable clinical logic to dynamically filter the gene-specific healthcare information.

The mapping database may be securely hosted on any suitable computing infrastructure. The suitable computing infrastructure may include a distributed server architecture. Alternatively, or additionally, the mapping database may be hosted on any one or more of the group including a centralised server network, a cloud-based computing environment, a hybrid-cloud infrastructure, a peer-to-peer network, and an on-premise dedicated hardware system. It is to be appreciated that hosting the mapping database on an on-premise dedicated hardware system may provide enhanced cryptographic security and compliance with localised data sovereignty regulations, whereas hosting on a cloud-based computing environment may provide dynamic scalability for the multi-phase computational mapping operation.

The system may include a knowledge base updating operation. The knowledge base updating operation may be configured to structurally integrate new data into the mapping database. The new data may correspond to contents of the mapping database. The knowledge base updating operation may be configured to receive the new data which forms part of an external source. The external source may be in the form of any one or more of the group including newly published pharmacogenomic guidelines, emerging clinical evidence, new clinical guidelines, emerging scientific evidence, and updated regulatory labelling. Structurally integrating the new data may include generating new discrete data elements and automatically assigning new primary keys and foreign keys, or generating new computational nodes and directed edges, within the mapping database. It is to be appreciated that integrating the new data alters the contents of the mapping database, thereby updating the machine-readable logical relationships and the computable clinical logic.

The multi-phase mapping operation may be initiated by the processor automatically upon successful execution of the data storing operation. Alternatively, the mapping operation may be initiated upon user selection of a selectable mapping element displayed via the user interface.

The multi-phase mapping operation may be executed in accordance with the computable clinical logic to generate individual-specific mapped data. The multiphase mapping operation may be configured to store the individual-specific mapped data against the individual profile.

The individual-specific mapped data may include any one or more mapped data groups including mapped phenotypic data, mapped pharmacogenetic data, mapped drug-drug interaction data, mapped pharmacogenomic data, mapped biological data, mapped pharmacological data, mapped risk data, mapped alternative treatment data, mapped healthcare data, and mapped condition-related healthcare data.

The mapped data groups may be computationally categorised into mapped database domains. The mapped database domains may overlap with each other. The mapped database domains may include mapped gene-related data, mapped medication-related data, and mapped healthcare-related data. The mapped gene-related data may include the mapped phenotypic data, the mapped biological data, the mapped pharmacogenetic data, and the mapped pharmacogenomic data. The mapped medication-related data may include the mapped pharmacological data, the mapped drug-drug interaction data, the mapped pharmacogenetic data, and the mapped pharmacogenomic data. The mapped healthcare-related data may include the mapped healthcare guidance data and the mapped condition-related data.

It is to be appreciated that the individual-specific mapped data may define a computationally filtered subset of the entire mapping database which is filtered by the individual-specific user data. The computationally filtered subset may include any data within the mapping database which is associated with the individual.

The individual-specific mapped data may further include derived computational inferences. The derived computational inferences may include any one or more of the group including derived pharmacogenomic information, derived pharmacogenetic information, computationally established genotype-phenotype correlations, genetic traits or phenotypes, disease-associated genetic markers, genetic predispositions, carrier status for inherited conditions, disease risk scores, polygenic risk scores, pathway analysis results, ancestry or ethnic origins, and genetic relatedness to other individuals.

The individual-specific mapped data may form the basis of the gene-specific healthcare information associated with the user. The individual-specific mapped data may form part of the output.

The multi-phase mapping operation may include a plurality of phases for generating the individual-specific mapped data. Execution of the plurality of phases may be initiated concurrently by the processor upon initiation of the multi-phase mapping operation. The processor may be configured to manage computational dependencies between the plurality of phases during concurrent execution. Managing the computational dependencies may include temporarily suspending execution of a dependent phase pending generation of a required computational variable by an antecedent phase. Managing the computational dependencies may further include automatically resuming execution of the dependent phase upon receipt of the required computational variable. It is to be appreciated that this dependency-aware concurrent execution ensures optimal processing efficiency while maintaining chronological data integrity across the multi-phase mapping operation.

The plurality of phases may include the gene-mapping phase.

The gene-mapping phase may include a phenotypic mapping operation for mapping the user genetic information to the phenotypic data stored on the mapping database. The phenotypic mapping operation may be executed by the processor. The phenotypic mapping operation may be configured to generate the mapped phenotypic data. The phenotypic mapping operation may be configured to computationally process and translate the user genetic information into the mapped phenotypic data. The mapped phenotypic data may form part of a phenotypic mapping output. The phenotypic mapping operation may include querying the mapping database using the user genetic information to extract corresponding observable characteristics associated with the user genetic information. The phenotypic mapping operation may include generating metabolic activity profiles based on a corresponding metabolic phenotype associated with the user genetic information. The metabolic activity profiles may define a computational representation of how the individual metabolises specific chemical compounds. The metabolic activity profiles may form part of the phenotypic mapping output. The phenotypic mapping operation may include storing the phenotypic mapping output against the individual profile. It is to be appreciated that raw user genetic information, such as specific gene alleles, typically lacks direct clinical actionability until computationally translated into the individual-specific mapped phenotypic data, thereby establishing a functional and machine-readable biological baseline for the individual.

The gene-mapping phase may include a pharmacogenetic mapping operation for mapping the user genetic information to the pharmacogenetic data stored on the mapping database. The pharmacogenetic mapping operation may be executed by the processor. The pharmacogenetic mapping operation may be configured to generate the mapped pharmacogenetic data. The mapped pharmacogenetic data may form part of a pharmacogenetic mapping output.

The pharmacogenetic mapping operation may include querying the mapping database using the user genetic information to extract corresponding drug reactions. The pharmacogenetic mapping operation may include generating pharmacogenetic reaction profiles based on the corresponding drug reactions associated with the user genetic information. The pharmacogenetic reaction profiles may define a computational representation of how the individual reacts to specific pharmaceutical active ingredients. The mapped pharmacogenetic reaction profiles may form part of the pharmacogenetic mapping output. The pharmacogenetic mapping operation may include storing the pharmacogenetic mapping output against the individual profile.

The gene-mapping phase may include a pharmacogenomic mapping operation for mapping the user genetic information to the pharmacogenomic data stored on the mapping database. The pharmacogenomic mapping operation may be executed by the processor. The pharmacogenomic mapping operation may be configured to generate the mapped pharmacogenomic data. The mapped pharmacogenomic data may form part of a pharmacogenomic mapping output. The pharmacogenomic mapping operation may include querying the mapping database using the user genetic information to extract corresponding likely drug reactions. In particular, the pharmacogenomic mapping operation may include querying the mapping database using the user genetic information to extract corresponding biological pathways and/or therapeutic classifications associated with active pharmaceutical ingredients and the user genetic information. The pharmacogenomic mapping operation may include generating mapped pharmacogenomic reaction profiles based on the corresponding biological pathways. The pharmacogenomic reaction profiles may define a computational representation of how the individual would likely react to specific pharmaceutical active ingredients. The mapped pharmacogenomic reaction profiles may form part of the pharmacogenomic mapping output. The pharmacogenomic mapping operation may include storing the pharmacogenomic mapping output against the individual profile.

The plurality of phases may include the medication-mapping phase.

The medication-mapping phase may include a drug-drug interaction mapping operation for mapping the user medication information to the drug-drug interaction data stored on the mapping database. The drug-drug interaction mapping operation may be executed by the processor. The drug-drug interaction mapping operation may be configured to generate the mapped drug-drug interaction data. The mapped drug-drug interaction data may form part of a drug-drug interaction mapping output. The drug-drug interaction mapping operation may include querying the mapping database using the user medication information to extract corresponding drug-drug contraindications. The drug-drug interaction mapping operation may include generating drug-drug interaction profiles based on the corresponding drug-drug contraindications. The drug-drug interaction profiles may define a computational representation of the specific drug-drug interactions taking place or having taken place historically, as a result of the user medication information of the individual. The drug-drug interaction profiles may form part of the drug-drug interaction mapping output. The drug-drug interaction mapping operation may include storing the drug-drug interaction mapping output against the individual profile.

The medication-mapping phase may include a pharmacological mapping operation for mapping the user medication information to the pharmacological data stored on the mapping database. The pharmacological mapping operation may be executed by the processor. The pharmacological mapping operation may be configured to generate the mapped pharmacological data. The mapped pharmacological data may form part of a pharmacological mapping output. The pharmacological mapping operation may include querying the mapping database using the user medication information to extract corresponding uses, effects and modes of action of specific drugs. The pharmacological mapping operation may include generating pharmacological profiles based on the corresponding uses, effects and modes of action of specific drugs. The pharmacological profiles may define a computational representation of the uses, effects and modes of actions of specific drugs associated with the user medication information. The pharmacological profiles may form part of the pharmacological mapping output. The pharmacological mapping operation may include storing the pharmacological mapping output against the individual profile.

The plurality of phases may include risk-mapping phase. The risk-mapping phase may include a risk mapping operation for mapping the user medication information and user genetic information to risk-related information stored on the mapping database. The risk mapping operation may be executed by the processor. The risk mapping operation may be configured to generate mapped risk data. The mapped risk data may form part of a risk evaluation output. The risk mapping operation may include a pharmacogenetic risk evaluation. The pharmacogenetic risk evaluation may include cross-referencing the user medication information with the metabolic activity profiles generated by the phenotypic mapping operation. The pharmacogenetic risk evaluation may be configured to identify a pharmacogenetic risk associated with the individual. The identified pharmacogenetic risk may form part of the mapped risk data. The risk evaluation operation may include a drug-drug interaction risk evaluation. The drug-drug interaction risk evaluation may include cross-referencing the user medication information against the drug-drug interaction data contained within the mapping database. The drug-drug interaction risk evaluation may be configured to identify a drug-drug interaction risk associated with the user medication information. The identified drug-drug interaction risk may form part of the mapped risk data. The risk evaluation operation may be configured to computationally map the identified risks to the mapped pharmacogenetic data and mapped drug-drug interaction data to generate the mapped risk data. The risk evaluation operation may include storing the risk evaluation output against the individual profile.

The risk mapping operation may be configured to execute the cross-referencing of the metabolic activity profiles generated by the phenotypic mapping operation and the cross-referencing of the drug-drug interaction data concurrently. It is to be appreciated that executing the pharmacogenetic risk evaluation and the drug-drug interaction risk evaluation concurrently enables the processor to detect complex, compounding adverse reactions. This computational concurrency is believed to provide a distinct technical advantage by dynamically processing multi-variable clinical interactions that are mathematically prohibitive for a human practitioner to calculate manually.

The plurality of phases may include an alternative-treatment-mapping-phase. The alternative-treatment-mapping phase may include an alternative treatment mapping operation for mapping the mapped risk data generated by the risk mapping operation to data contained in the mapping database relating to alternative treatments. The alternative treatment mapping operation may be executed by the processor. The alternative treatment mapping operation may be initiated upon identification of the pharmacogenetic risk or the drug-drug interaction risk by the risk mapping operation. The alternative treatment mapping operation may be configured to computationally query the mapping database to extract an alternative treatment suggestion associated with the identified risk. The alternative treatment mapping operation may be configured to generate the mapped alternative treatment data based on the extracted alternative treatment suggestion. The mapped alternative treatment data may form part of an alternative treatment output. The alternative treatment mapping operation may include storing the alternative treatment output against the individual profile. It is to be appreciated that extracting the alternative treatment suggestion computationally bridges the gap between risk identification and clinical resolution. The alternative treatment extraction is believed to ensure the system functions as a proactive, automated guidance engine rather than a passive alert mechanism, dynamically presenting the health practitioner with safe, computationally verified treatment pathways.

The plurality of phases may include a biological-mapping phase. The biological mapping phase may include a biological mapping operation for mapping the user genetic information, the user medication information and the user health condition information to the biological data stored on the mapping database. The biological mapping operation may be executed by the processor. The biological mapping operation may be configured to generate mapped biological data. The mapped biological data may form part of a biological mapping output. The biological mapping operation may include querying the mapping database using the user genetic information, the user medication information and the user health condition information to extract corresponding biological data. The biological mapping operation may include generating biological reaction profiles based on the corresponding biological data. The biological reaction profiles may define a computational representation of biological reactions experienced and/or likely to be experienced by the individual based on the user genetic information, the user medication information and the user health condition information. The biological reaction profiles may form part of the biological mapping output. The biological mapping operation may include storing the biological mapping output against the individual profile.

The plurality of phases may include a healthcare-mapping phase. The healthcare-mapping phase may include a healthcare mapping operation for mapping any one or more system operation outputs to the healthcare data stored on the mapping database.

The system operation outputs may include the phenotypic mapping output, the pharmacogenetic mapping output, the drug-drug interaction mapping output, the pharmacogenomic mapping output, the biological mapping output, the pharmacological mapping output, the risk evaluation output, and the alternative treatment output.

The healthcare mapping operation may be executed by the processor. The healthcare mapping operation may be configured to generate the mapped healthcare data. The mapped healthcare data may form part of a healthcare mapping output.

The healthcare mapping operation may include querying the mapping database using any one or more of the system operation outputs to extract corresponding actionable clinical recommendations. The healthcare mapping operation may include generating a healthcare guidance profile based on the corresponding actionable clinical recommendations. The healthcare guidance profile may define a computational representation of the specific actionable clinical recommendations for the individual based on any one or more of the system operational outputs. The healthcare guidance profile may form part of the healthcare guidance mapping output. The healthcare guidance profile may form part of the gene-specific healthcare guidance information. The healthcare guidance mapping operation may include storing the healthcare mapping output against the individual profile.

The plurality of phases may include a health-condition-mapping phase. The health-condition-mapping phase may include a condition mapping operation for mapping the user health condition information to the individual-specific mapped data. In particular, the condition mapping operation may be configured to map the user health condition information to the mapped gene-related data, the mapped medication-related data, and the mapped healthcare-related data to generate mapped condition-related healthcare data associated with the individual to be included in the output. It is to be appreciated that the mapped condition-related healthcare data pertains to a specific health condition of the individual and that the mapped condition-related healthcare data can be used to filter all the individual-specific mapped data to easily obtain the mapped data most relevant to the specific health condition.

The system may include an output classification operation. The output classification operation may be executed by the processor. The output classification operation may be configured to computationally process and categorise any one or more of the system operation outputs into a plurality of classification categories. The plurality of classification categories may be indicative of varying levels of clinical risk. The plurality of classification categories may be selected from any of the group including standard administration, therapeutic caution, and alternative treatment recommended. The output classification operation may be configured to assign a distinct visual identifier to each of the classification categories. The distinct visual identifier may include a colour-coded classification. The output classification operation may be configured to compile the categorised system operation outputs and the assigned visual identifiers to generate a classification profile. The classification profile may form part of the gene-specific healthcare guidance. The output classification operation may include storing the classification profile against the individual profile. It is to be appreciated that the output classification operation computationally structures the complex biological and pharmacological data into a visually discernible hierarchy. This structural categorisation is believed to minimise the cognitive load on the health practitioner, facilitating rapid, computationally assisted clinical decision-making without requiring the practitioner to manually interpret the underlying raw data.

The system may include a dynamic re-mapping operation. The dynamic re-mapping operation may be configured to automatically re-execute the multi-phase mapping operation. The dynamic re-mapping operation may be initiated upon detection of a system variable update. The system variable update may be classified as either a local state modification or a global state modification. The local state modification may include a modification to the individual-specific user data, preferably the user medication data or the user health condition data. The global state modification may include an update or modification to the contents of the mapping database, preferably after execution of the knowledge base updating operation. It is to be appreciated that the dynamic re-mapping operation ensures that the individual-specific mapped data remains continuously accurate, automatically generating updated healthcare guidance information without requiring manual practitioner initiation whenever the individual changes medications, updates any user health condition data or whenever new data is integrated into the system.

The dynamic re-mapping operation may be configured to computationally filter a list of active pharmaceutical ingredients according to the current user medication data or the current user health condition data. It is to be appreciated that because the current user medication data or the current user health condition data may be updated dynamically by the health practitioner during a clinical consultation, the processor executes the dynamic re-mapping operation to output a dynamically filtered, real-time clinical assessment based on the most recent health status of the individual.

The system may include an authorisation operation for enabling the individual to authorise the health practitioner to access the individual profile, thereby assisting the health practitioner to provide the gene-specific healthcare guidance information to the individual. The authorisation operation may be executed by the processor to securely manage access to the repository. The authorisation operation may include displaying a login visual to a user via the user interface. The user may include the individual or a health practitioner. The login visual may include data input fields configured to receive the login details of the user. The authorisation operation may include a multi-factor authentication module. The multi-factor authentication module may be configured to receive a one-time pin (OTP) or a time-based one-time pin (TOTP) to successfully authenticate the user.

The processor may be configured to execute the authorisation operation via a plurality of access interfaces. The plurality of access interfaces may include a dedicated client application executable on a personal computing device associated with the user. Alternatively, the plurality of access interfaces may include a web-based application accessible via an internet browser.

The authorisation operation may include a practitioner-initiated access protocol. The practitioner-initiated access protocol may be initiated by a registered health practitioner submitting an access request associated with a specific registered individual. The processor may be configured to transmit the access request to a personal computing device associated with the individual. The access request may include identification information of the requesting health practitioner and a computational prompt configured to receive an access grant or an access denial from the individual. Upon receiving the access grant, the processor may be configured to generate an access token, such as a TOTP, for the health practitioner to input via the user interface. The practitioner-initiated access protocol may be configured to computationally restrict access to the individual's profile to a predefined time limit.

The authorisation operation may include an individual-initiated access protocol. The individual-initiated access protocol may be configured to display a directory of registered health practitioners to the individual. The individual-initiated access protocol may be configured to receive a selection of a specific registered health practitioner from the directory. Upon receiving the selection, an access notification including a secure access link may be generated and transmitted to a computing device associated with the selected health practitioner.

The authorisation operation may include an administrative access protocol. The administrative access protocol may be configured to permit a system service provider to securely access the repository. The administrative access protocol enables the system service provider to computationally alter or update system variables to maintain continuous system operability.

The processor may be configured to execute a plurality of integrated computational modules. The integrated computational modules may be initiatable by the health practitioner. The integrated computational modules may include any one or more of the group including a query module, a dual-view rendering engine, a longitudinal medication history module, and a comprehensive variant access module.

The query module may be configured to receive a manual query input from the health practitioner via the user interface. The manual query input may include any one or more of the group including a proposed active pharmaceutical ingredient, a therapeutic classification, and an identified clinical indication. The query module may be configured to computationally execute a simulated evaluation of the manual query input against the individual-specific mapped data prior to prescription. The simulated evaluation may include dynamically filtering contents of the mapping database based on user genetic information, specific user medication data, and specific user health condition data of the individual. It is to be appreciated that the query module enables predictive scenario testing, allowing the health practitioner to computationally verify the safety of a proposed treatment or instantly generate a list of genotype-compatible alternatives within a specific therapeutic class without permanently modifying the core user medication data.

The dual-view rendering engine may be configured to structurally format a graphical user interface. The dual-view rendering engine may be configured to computationally toggle the graphical user interface between a clinical summary interface and an evidence-based interface. The clinical summary interface may be configured to display mapped structured clinical action mappings and mapped distinct visual identifiers obtained from the output of the multi-phase mapping operation. The evidence-based interface may be configured to display mapped scientific source data and mapped traceable reference links obtained from the output of the multi-phase mapping operation. It is to be appreciated that the dual-view rendering engine dynamically manages the cognitive load on the health practitioner by separating rapid-decision data from deep-dive scientific verification data, thereby improving visual interpretability while preserving the underlying integrity of system-generated outputs.

The longitudinal medication history module may be configured to chronologically aggregate the historical user medication data. The longitudinal medication history module may retrospectively execute the risk mapping operation on the historical user medication data using the individual-specific mapped phenotypic data. It is to be appreciated that retrospectively mapping past medications against the newly translated phenotypic baseline allows the processor to computationally identify root causes of historical adverse drug reactions or prior treatment failures.

The comprehensive variant access module may be configured to provide structured access to raw user genetic information. The comprehensive variant access module may be configured to display specific gene alleles and genotypic markers independent of the individual-specific mapped phenotypic data. The raw user genetic information may include information regarding genetic variants beyond those currently implicated in active clinical recommendations. It is to be appreciated that the comprehensive variant access module accommodates deep-dive specialist review, providing the health practitioner with transparent access to underlying genetic architecture of the individual.

It is to be appreciated that while the mapping operation functions as an automated processing pipeline, the integrated computational modules provide the health practitioner with specific, on-demand digital tools to manually interrogate the data and visually customise the gene-specific healthcare guidance.

The system may include a reporting operation executable by the processor. The reporting operation may be configured to computationally compile the individual-specific mapped data and the gene-specific healthcare guidance into a secure digital document, such as a portable document format (PDF) report.

The reporting operation may include a summary reporting operation. The summary reporting operation may be configured to generate a summary report. The summary report may be structurally restricted to the current user health condition data and/or the current user medication data of the individual. The summary reporting operation may be configured to filter the individual-specific mapped data to display pharmacogenetic insights, identified pharmacogenetic risks, and specific alternative treatment suggestions solely associated with a current medication regimen of the individual. It is to be appreciated that the summary report provides a targeted, highly actionable snapshot optimised for immediate clinical decision-making regarding the active pharmaceutical ingredients currently prescribed to the individual.

The reporting operation may include a comprehensive reporting operation. The comprehensive reporting operation may be configured to generate a comprehensive report. The comprehensive report may include the complete output of the multi-phase mapping operation, and an output of any further system operations. Alternatively, the comprehensive report may include a complete computational mapping across a comprehensive list of active pharmaceutical ingredients. The comprehensive report may detail all potential gene-medicine interactions, pharmacogenomic pathways, and risk stratification classifications associated with the foundational genotypic data of the individual, independent of the current medication regimen.

The summary report and the comprehensive report may include the distinct visual identifiers, cautionary clinical flags, and structured clinical action mappings to rapidly convey therapeutic efficacy and potential risks. The reporting operation may be configured to embed a clinical audit trail within the reports. The clinical audit trail may include the traceable reference links and the curated scientific source data. It is to be appreciated that embedding the clinical audit trail allows the health practitioner to transparently view and verify the structured, rule-based clinical logic utilised by the processor to reach a particular recommendation or alternative treatment suggestion.

The reporting operation may be dynamically responsive to the dynamic re-mapping operation. The processor may be configured to automatically regenerate the summary report and the comprehensive report upon detection of a system variable update. It is to be appreciated that the dynamic regeneration ensures that any exported digital document remains continuously aligned with the most recent health status of the individual and latest global clinical guidelines.

The system may include a notification operation executable by the processor. The notification operation may be computationally linked to the dynamic re-mapping operation. The notification operation may be initiated upon execution of the dynamic re-mapping operation resulting specifically from a global state modification. The notification operation may include computationally comparing newly generated individual-specific mapped data against historically stored individual-specific mapped data. The notification operation may include computationally detecting a clinical deviation between the newly generated individual-specific mapped data and the historically stored data. The clinical deviation may indicate that the newly integrated scientific source data alters or influences a previously generated actionable clinical recommendation. Upon detecting the clinical deviation, the notification operation may be configured to dynamically generate a targeted clinical alert. The notification operation may be configured to automatically transmit the targeted clinical alert to the individual or the authorised health practitioner via the plurality of access interfaces or personal computing devices associated with the individual or health practitioner. It is to be appreciated that the notification operation transforms the system from a passive reference tool into a proactive, state-aware clinical surveillance engine, ensuring that all historical patient mappings are continuously and automatically audited against the latest scientific literature without requiring manual practitioner initiation.

According to a second aspect of the invention, there is provided a system for computationally generating gene-specific healthcare guidance information, the system including:
a processor with access to a memory containing instructions executable by the processor to perform operations including:
a data acquisition operation for acquiring individual-specific user data from an individual; and
a computational mapping operation for mapping the individual-specific user data to healthcare guidance data contained in a mapping database to generate an output including mapped gene-specific healthcare guidance data associated with the individual.

According to a third aspect of the invention, there is provided a system for computationally generating gene-specific healthcare guidance information, the system including:
a processor with access to a memory containing instructions executable by the processor to perform operations including:
a data acquisition operation for acquiring individual-specific user data from an individual, the individual-specific user data including user genetic information, and user medication information, and user health condition information; and
a computational mapping operation for mapping the individual-specific user data to healthcare guidance data contained in a mapping database to generate an output including mapped gene-specific healthcare guidance information associated with the individual.

According to a fourth aspect of the invention, there is provided a system for computationally generating gene-specific healthcare guidance information, the system including:
a processor with access to a memory containing instructions executable by the processor to perform operations including:
a data acquisition operation for acquiring individual-specific user data from an individual, the individual-specific user data including user genetic information, user medication information, and user health condition information; and
a multi-phase computational mapping operation for mapping the individual-specific user data to healthcare guidance data contained in a mapping database to generate an output including gene-specific healthcare guidance information associated with the individual, the multi-phase computational mapping operation including:
a gene-mapping phase for mapping the user genetic information to gene-related data contained in the mapping database to generate mapped gene-related data associated with the individual, the gene-related data including phenotypic data, pharmacogenetic data, and biological data;
a medication-mapping phase for mapping the user the medication information to medication-related data contained in the mapping database to generate mapped medication-related data associated with the individual, the medication-related data including the pharmacogenetic data, drug-drug interaction data, the biological data, and pharmacological data;
a healthcare-mapping phase for mapping the mapped gene-related data and the mapped medication-related data to healthcare-related data contained in the mapping database to generate mapped healthcare data associated with the individual to be included in the output.

According to a fifth aspect of the invention, there is provided a system for computationally generating gene-specific healthcare guidance information, the system including:
a processor with access to a memory containing instructions executable by the processor to perform operations including:
a data acquisition operation for acquiring individual-specific user data from an individual, the individual-specific user data including user genetic information, user medication information, and user health condition information; and
a multi-phase computational mapping operation for mapping the individual-specific user data to healthcare guidance data contained in a mapping database to generate an output including gene-specific healthcare guidance information associated with the individual, the multi-phase computational mapping operation including:
a gene-mapping phase for mapping the user genetic information to gene-related data contained in the mapping database to generate mapped gene-related data associated with the individual, the gene-related data including phenotypic data, pharmacogenetic data, and biological data;
a medication-mapping phase for mapping the user medication information to medication-related data contained in the mapping database to generate mapped medication-related data associated with the individual, the medication-related data including the pharmacogenetic data, drug-drug interaction data, the biological data, and pharmacological data;
a healthcare-mapping phase for mapping the mapped gene-related data and the mapped medication-related data to healthcare-related data contained in the mapping database to generate mapped healthcare data associated with the individual to be included in the output; and
a health-condition-mapping phase for mapping the user health condition information to the mapped gene-related data, the mapped medication-related data, and the mapped healthcare-related data to generate mapped condition-related healthcare data associated with the individual to be included in the output.

According to a sixth aspect of the invention, there is provided a system for computationally generating gene-specific healthcare guidance information, the system including:
a processor with access to a memory containing instructions executable by the processor to perform operations including:
a profile creation operation for creating profiles for users of the system and storing the profiles on a repository, the profile creation operation being configured to create an individual profile for a user in the form of an individual and to create a practitioner profile for a user in the form of a health practitioner.
a data acquisition operation for acquiring individual-specific user data from an individual, and storing the individual-specific user data against the individual profile, the individual-specific user data including user genetic information, user medication information, and user health condition information; and
a multi-phase computational mapping operation for mapping the individual-specific user data to healthcare guidance data contained in a mapping database to generate an output including mapped gene-specific healthcare guidance information associated with the individual, the multi-phase computational mapping operation including:
   a gene-mapping phase for mapping the user genetic information to gene-related data contained in the mapping database to generate mapped gene-related data associated with the individual;
   a medication-mapping phase for mapping the user medication information to medication-related data contained in the mapping database to generate mapped medication-related data associated with the individual;
   a healthcare-mapping phase for mapping the mapped gene-related data and the mapped medication-related data to healthcare-related data contained in the mapping database to generate mapped healthcare data associated with the individual to be included in the output.
an authorisation operation for enabling the individual to authorise the health practitioner to access the individual profile, thereby assisting the health practitioner to provide the gene-specific healthcare guidance information to the individual.

### BRIEF DESCRIPTION OF THE DRAWINGS

A genetic health information system in accordance with the invention will now be described by way of the following, non-limiting examples with reference to the accompanying drawings.

In the drawings:

FIG. 1 is a flow diagram providing a general overview of the genetic healthcare guidance system;

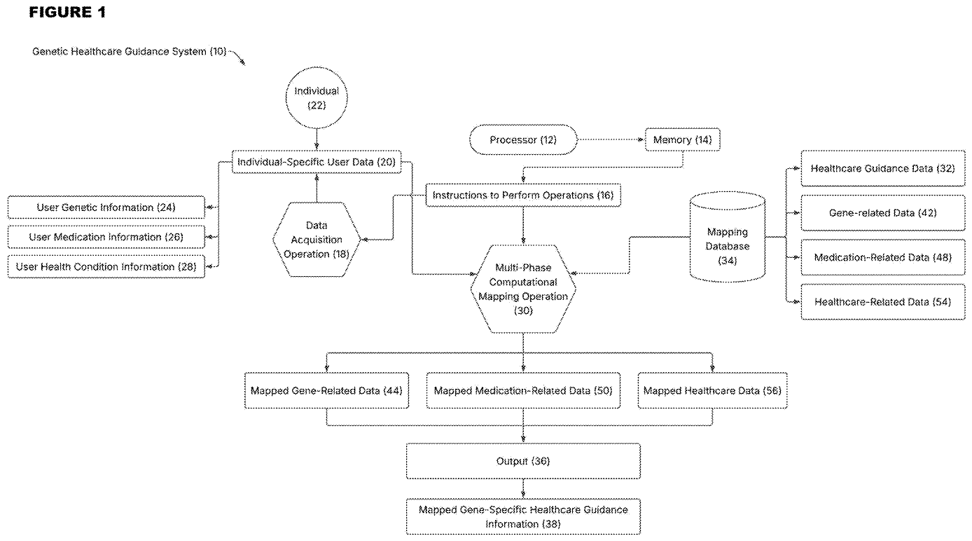

FIG. 2 is a flow diagram visually representing certain operations of the system;

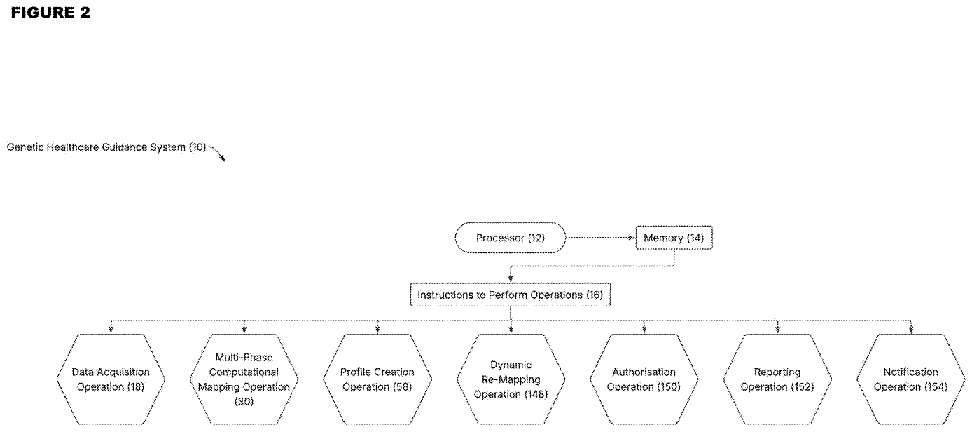

FIG. 3 is a flow diagram visually representing the operations of the multi-phase computational mapping operation; and

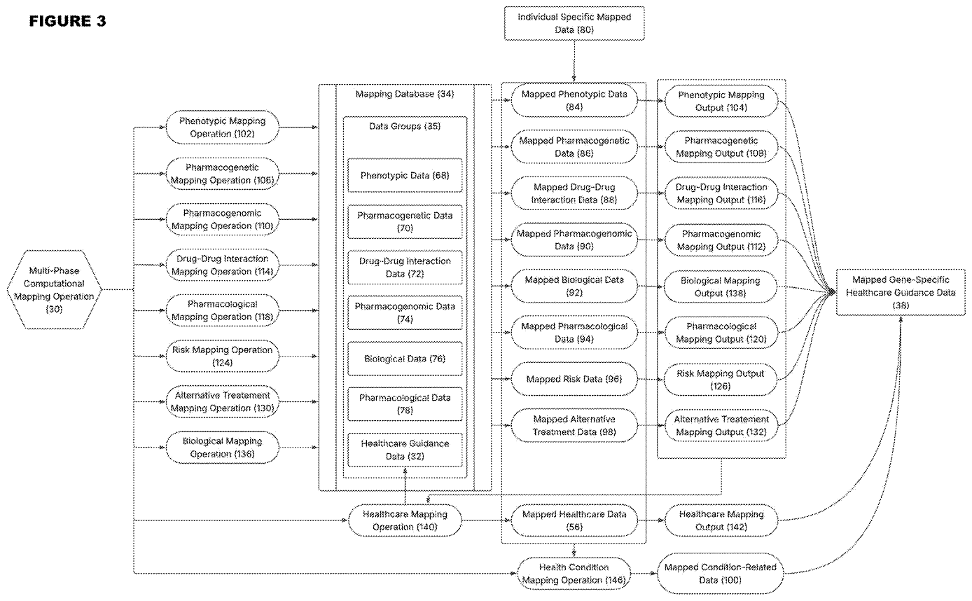

FIG. 4 is a flow diagram visually representing the profile creation operation.

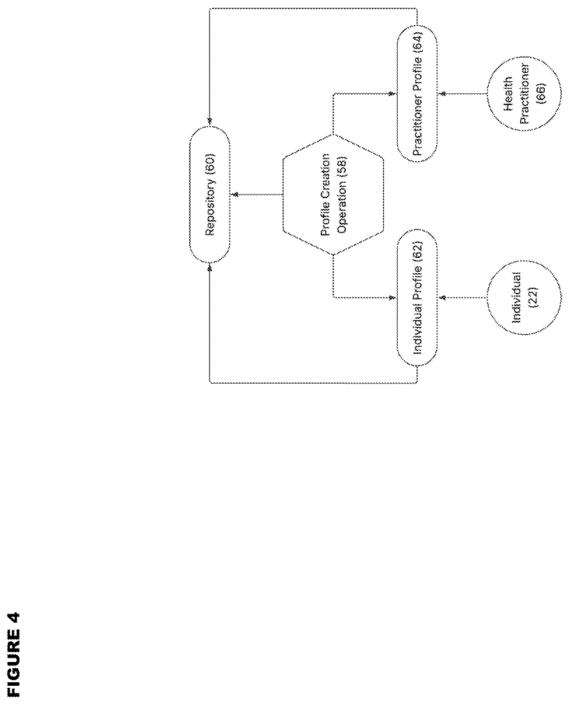

### DETAILED DESCRIPTION OF THE INVENTION

Referring to the accompanying figures, reference numeral 10 refers generally to a genetic healthcare guidance system. The genetic healthcare guidance system includes:
a processor 12 with access to a memory 14 containing instructions 16 executable by the processor 12 to perform operations including:
a data acquisition operation 18 for acquiring individual-specific user data 20 from an individual 22, the individual-specific user data 20 including user genetic information 24, user medication information 26, and user health condition information 28; and
a multi-phase computational mapping operation 30 for mapping the individual-specific user data 20 to healthcare guidance data 32 contained in a mapping database 34 to generate an output 36 including mapped gene-specific healthcare guidance information 38 associated with the individual 22, the multi-phase computational mapping operation 30 including:
a gene-mapping phase for mapping the user genetic information 24 to gene-related data 42 contained in the mapping database 34 to generate mapped gene-related data 44 associated with the individual 22;
a medication-mapping phase for mapping the user medication information 26 to medication-related data 48 contained in the mapping database 34 to generate mapped medication-related data 50 associated with the individual 22; and
a healthcare-mapping phase for mapping the mapped gene-related data 44 and the mapped medication-related data 50 to healthcare-related data 54 contained in the mapping database 34 to generate mapped healthcare data 54 associated with the individual 22 to be included in the output 36.

A display device is provided for receiving and displaying visual output data generated by the processor 12. The display device is accessible and controllable by the processor 12. The display device includes any suitable hardware configured to display the visual output data. The suitable hardware includes any of the group including a computer monitor, a mobile device screen, and a tablet display.

The processor 12 includes any suitable hardware device which is configured to execute the instructions 16. The hardware device includes any one or more of the group including a processing unit, micro-processor, and microcontroller. The hardware device forms part of a plurality of interconnected processing units. The plurality of interconnected processing units forms a distributed computing network or a cloud-based server architecture. The processor 12 is configured to output a user interface to the display device. The user interface is configured to facilitate user interaction with the system. The user interface includes any one or more of the group including a graphical user interface (GUI), a web-based portal, and a dedicated application interface.

The memory 14 includes a non-transitory computer-readable storage medium within which the instructions 16 are stored. The memory 14 is arranged in communication with the processor 12 to enable execution of the instructions 16 by the processor 12. The storage medium is further configured to host one or more databases or repositories for securely storing system data.

The instructions 16 are in the form of computer-readable instructions 16 for performing the operations. In particular, the instructions 16 are in the form of executable computer code. The computer code is logically structured as a plurality of distinct software modules or integrated into a unified computational framework to execute the operations.

The system includes a profile creation operation 58 for creating profiles for users of the system 10 and storing the profiles on a repository 60. The profile creation operation 58 is configured to create an individual profile 62 for a user in the form of an individual 22. The profile creation operation 58 is configured to create a practitioner profile 64 for a user in the form of a health practitioner 66.

The profile creation operation 58 includes a credential acquisition operation for acquiring credentials from the user. The credential acquisition operation is initiated upon user selection of a selectable profile creation element.

The credential acquisition operation includes displaying a credential acquisition visual to the user via the user interface. The credential acquisition visual includes a plurality of credential capture elements configured to receive the credentials from the user. The plurality of credential capture elements includes any one or more of the group including a manual text entry field, a file upload interface, and an external database integration module. The file upload interface is configured to receive electronic documents containing the credentials. The external database integration module is configured to establish a communication link with a remote credential database to automatically extract the credentials.

The credentials include login details. The login details include a unique identifier and an authenticator. The unique identifier is in the form of any of the group including a name, username, full name, identification number, email address, telephone number, cellular number. The authenticator is in the form of any of the group including a password, code, OTP (one-time-pin), personal identification number (PIN), passcode, time-based one-time password (TOTP), biometric input, fingerprint scan, facial recognition data, iris scan, voiceprint, hardware security token, software security token, cryptographic key, authentication link, spatial pattern, and security question response.

The credentials include user type information. The user type information includes a user type identifier. The user type identifier is an identifier of a user type. The user type includes individual type and health practitioner type. Input of the user type identifier serves to enable the system to differentiate between individuals 22 and health practitioners 66.

The profile creation operation 58 includes an allocation operation for allocating a dedicated memory space within the repository 60 to the user. The profile creation operation 58 includes a credential storing operation for storing the credentials within the dedicated memory space. The profile creation operation 58 includes a profile identifier generation operation for generating a unique profile identifier and assigning the unique profile identifier to the dedicated memory space. The allocation operation, credential storing operation and profile identifier generation operation is initiated upon user selection of a selectable confirmation element displayed to the user within the credential acquisition visual. It is to be appreciated that the profiles are defined by the unique profile identifiers and contents of the memory space dedicated to the users. The profiles are configured to represent distinct digital data records for the individuals 22 and health practitioners 66, respectively.

The profile creation operation 58 is configured to establish registered users, wherein the registered users include individuals 22 and health practitioners 66 having dedicated profiles stored on the repository 60.

It is to be appreciated that generating a distinct health practitioner profile 64 provides the technical advantage of enabling role-based access control and secure data segregation within the system 10. In particular, utilising a separate health practitioner profile 64 allows the system to execute isolated authentication processes and manage targeted access permissioning independently of the individual profile 62, thereby reducing data duplication and enhancing the secure handling of sensitive clinical information.

The repository 60 is accessible by the processor 12. The repository 60 forms part of a remote user database. The remote user database is in the form of a cloud-based database.

The individual 22 includes any human subject. The human subject includes a primary user of the system seeking healthcare guidance. The primary user includes any one or more of the group including a patient, a prospective patient, a consumer, and a research participant. The individual 22 includes a representative acting on behalf of the primary user. The representative includes any one or more of the group including a legal guardian, a parent, a caregiver, and a designated proxy. The designated proxy is authorised to create and manage the individual profile 62 on behalf of a dependent. The dependent includes a minor child or a legally incapacitated adult.

The health practitioner 66 is in the form of any person engaged in human health as a profession. In particular the health practitioner 66 is in the form of any one or more of the group including, but not limited to, a medical doctor, physician, dietician, optometrist, ophthalmologist, pharmacist, physical therapist, occupational therapist, dentist, psychologist, psychiatrist, sports trainer, genetic specialist, pathologist, nurse practitioner, midwife, chiropractor, speech therapist, audiologist, radiologist, anaesthesiologist, dermatologist, neurologist, cardiologist, pulmonologist, gastroenterologist, endocrinologist, urologist, orthopaedic surgeon, oncologist, rheumatologist, geriatrician, paediatrician, nutritionist, immunologist, allergist, naturopath, homeopath, acupuncturist, and reflexologist.

The data acquisition operation 18 includes storing the individual-specific user data 20 against the individual profile 62. The data acquisition operation 18 includes displaying a user data acquisition visual to the user via the user interface. The user data acquisition visual includes a plurality of data capture elements configured to receive the individual-specific user data 20. The plurality of data capture elements includes any one or more of the group including a manual text entry field, a file upload interface, and an external database integration module. The file upload interface is configured to receive electronic documents containing the individual-specific user data 20. The external database integration module is configured to establish a communication link with a remote user information database to automatically extract the individual-specific user data 20.

The data acquisition operation 18 further includes a dynamic data updating operation. The dynamic data updating operation is executed asynchronously to continuously maintain the individual-specific user data 20. The dynamic data updating operation is configured to permit manual updating of the individual-specific user data 20 by an authorised third party. The authorised third party includes an authorised health practitioner 66 or an authorised medical aid service provider. The manual updating includes updating the user medication information 26 and the user health condition information 28 to reflect current drug prescriptions, identified symptoms, medical test results, or the presence of illness subsequent to a clinical consultation.

The dynamic data updating operation is configured to permit automatic updating of the individual-specific user data 20. The automatic updating is executed by the external database integration module. The automatic updating includes automatically receiving continuous health-related data from a network-connected smart device associated with the individual 22. The automatic updating further includes securely querying an external database associated with the medical aid service provider to automatically extract updated user medication information 26 and/or user health condition information 28. It is to be appreciated that executing the dynamic data updating operation ensures the system continuously reflects the current health status of the individual 22, thereby computationally triggering dynamic re-mapping operations in response to newly acquired user medication information 26 and/or user health condition information 28.

The individual-specific user data 20 includes user identification information. The user identification information includes any one or more of the group including identification number, name, surname, full names, username, and residential address. The identification information includes contact information. The contact information includes any one or more of the group including email address, mobile number, and contact details of third parties. The third parties include any of the group including family members, legal guardians, and friends.

The individual-specific user data 20 includes the user genetic information 24. The user genetic information 24 includes foundational genotypic data of the individual. The foundational genotypic data includes uninterpreted biological data. The uninterpreted biological data includes any one or more of the group including genetic variants, single nucleotide polymorphisms (SNPs), specific genetic alleles, gene expression profiles, chromosomal abnormalities, DNA methylation patterns, epigenetic modifications, copy number variations, mitochondrial DNA data, haplogroups, structural variations, non-coding RNA analysis, carrier status for inherited conditions, and ancestry or ethnic origins. The user genetic information 24 further includes genetic testing results. The genetic testing results include raw genotyping data and raw sequencing data. The user genetic information 24 includes a digital genetic file. The digital genetic file includes an output file generated by a third-party genetic testing laboratory. The user genetic information 24 further includes historical user inputs. The historical user inputs include familial genetic history and genetic counselling records. It is to be appreciated that the user genetic information 24 is obtained via a processed DNA sample of the user. The DNA sample may typically be in the form of a buccal swab or a blood sample, preferably being in the form of a buccal swab. It is to be appreciated further that the processed DNA sample typically produces genetic data capable of being stored on the repository 60 against the individual profile 62.

The individual-specific user data 20 includes the user medication information 26. The user medication information 26 includes historical and/or current user medication information. The user medication information 26 includes information relating to any one or more of the group including medications, prescribed drugs, dietary supplements, herbal remedies, and alternative medicine usage.

The individual-specific user data 20 includes the user health condition information 28. The user health condition information 28 includes historical and/or current user health condition data. The user health condition information 28 includes any health-related information which may influence or relate to a health condition of a human being. The health-related information includes any one or more of the group including medical concerns, illnesses, diagnoses, treatments, previous medical practitioners, family medical history, sleep quality, stress levels, exercise regimes, allergies, known adverse drug reactions, vaccination history, surgical procedures, wound healing progress, surgical recovery status, lifestyle habits, dietary information, mental health history, cognitive function, reproductive health, pregnancy or lactation status, laboratory test results, renal function indicators, hepatic function indicators, imaging reports, vital signs (including blood pressure, blood oxygen levels, heart rate, body temperature, and respiratory rate), biometric data (including weight, height, and body mass index), blood type, immunisations, social determinants of health, substance and alcohol use history, environmental exposures, preventive screenings, therapeutic interventions, rehabilitation progress, chronic conditions, physical disabilities, implanted or wearable medical devices, patient-reported outcomes, and organ donation preferences. The user health condition information 28 further includes occupational data relating to the nature of the user's employment, preferably including a measure of a degree of stress experienced by users in a particular employment type.

The individual-specific user data 20 includes user activity data. The user activity data includes historical and/or current user activity data. The user activity data includes any one or more of the group including a historical record of overall measurement of activity and/or inactivity, steps, heart rate, exercises, training sessions, specific sports associated with the user, duration of physical activities, calories burned, distance covered, average pace or speed, workout intensity levels, rest periods, sleep patterns, sedentary time, active time, exercise goals and achievements, training plans, fitness assessments, heart rate variability, recovery metrics, exercise equipment usage, workout classes attended, personal bests, performance metrics, exercise-related injuries, exercise frequency, exercise preferences, and training load.

The individual-specific user data 20 includes user medical aid data. The user medical aid data includes historical and/or current user medical aid data. The user medical aid data includes any of the group including medical aid name, medical aid type, medical aid number, medical aid provider, medical aid scheme name, plan option, benefit tier, policy number, primary member identifier, dependent code, beneficiary number, principal member details, coverage limits, benefit exclusions, available medical savings balance, co-payment requirements, excess parameters, claims history, pre-authorisation records, coverage inception date, coverage expiration date, and funding availability.

While the individual-specific user data 20 includes any combination of the aforementioned data categories, execution of the multi-phase mapping operation 30 specifically relies upon the acquisition of at least the user genetic information 24 and the user medication information 28. Accordingly, the individual-specific user data 20 includes at least the user genetic information 24 and the user medication information 26.

The data acquisition operation 18 includes a data storing operation for storing acquired individual-specific user data 20 against the individual profile 62. The data storing operation is initiated upon receipt of the individual-specific user data 20 via the data capture elements. Storing the individual-specific user data 20 against the individual profile 62 includes writing the individual-specific user data 20 into the dedicated memory space allocated to the individual 22. The data storing operation includes associating the acquired individual-specific user data 20 with the unique profile identifier assigned to the dedicated memory space. It is to be appreciated that associating the acquired individual-specific user data 20 with the unique profile identifier ensures structural database integrity and enables the processor 12 to accurately retrieve the individual-specific user data 20 during the multi-phase mapping operation 30.

The data acquisition operation 18 is executed sequentially and immediately following the profile creation operation 58 within a continuous individual onboarding session. In such an embodiment, the processor 12 is configured to display the data acquisition visual automatically upon creation of the individual profile 62. The data acquisition operation 18 is executed asynchronously at any time subsequent to the profile creation operation 58 upon successful authentication of the individual 22 using the login details.

The system 10 includes a practitioner verification operation for verifying a professional status of the health practitioner 66. The practitioner verification operation is initiated by the processor 12 upon determining that the user type identifier corresponds to the health practitioner 66.

The practitioner verification operation includes a practitioner data acquisition operation. The practitioner data acquisition operation includes displaying a verification visual to the health practitioner 66 via the user interface. The verification visual includes a plurality of data capture elements configured to receive practitioner-specific user data.

The practitioner-specific user data includes any one or more of the group including field of practice, associated governing bodies, professional credentials, license identifier, practice identifier, regulatory registration details, national healthcare provider identifier, medical board license number, prescribing identifier, controlled substance registration number, professional council registration number, board certification identifier, specialisation classification code, national or regional registry number, professional association membership number, health facility identification number, and digital practitioner certificate.

The practitioner data acquisition operation is executed sequentially and immediately following the profile creation operation 58 within a continuous practitioner onboarding session. In such an embodiment, the processor 12 is configured to display the verification visual automatically upon creation of the practitioner profile 64. The practitioner data acquisition operation is executed asynchronously at any time subsequent to the profile creation operation 58 upon successful authentication of the health practitioner 66 using the login details.

The practitioner verification operation further includes a practitioner data validation operation. The practitioner data validation operation includes computationally comparing the received practitioner-specific user data against a verification database. The verification database includes a remote regulatory database accessible by the processor 12. The computational comparison includes transmitting an automated query to the remote regulatory database, the query containing at least a portion of the practitioner-specific user data.

The practitioner data validation operation further includes receiving a validation response from the remote regulatory database. The validation response includes a verification confirmation or a verification denial. The practitioner data validation operation includes updating a verification status of the health practitioner profile 64 based on the received validation response. Updating the verification status includes assigning a verified state to the dedicated memory space associated with the health practitioner 66 upon receipt of the verification confirmation.

The system 10 includes a use prohibition operation for prohibiting performance of the authorisation operation if the professional status of the health practitioner 66 is not verified. The use prohibition operation is initiated by the processor 12 upon receipt of the verification denial or upon determining that the dedicated memory space associated with the health practitioner 66 lacks the verified state. Prohibiting performance of the authorisation operation includes technical enforcement by the processor 12. On a user interface level, the technical enforcement includes disabling or hiding one or more selectable elements associated with the authorisation operation on the display device. On a database level, the technical enforcement includes the processor 12 restricting read and write access to the dedicated memory space defining the individual profile 62. It is to be appreciated that ensuring that only verified medical professionals may use the system prevents unverified practitioners or unauthorised nodes from being granted access to sensitive and/or confidential information stored against individual profiles 62, thereby maintaining cryptographic and structural data security.

The mapping database 34 is accessible by the processor 12.

The mapping database 34 includes any one or more data groups 35 including phenotypic data 68, pharmacogenetic data 70, drug-drug interaction data 72, pharmacogenomic data 74, biological data 76, pharmacological data 78, and healthcare guidance data 32.

The phenotypic data 68 includes a plurality of observable characteristics structurally linked to specific gene variations. The plurality of observable characteristics includes metabolic phenotypes. The metabolic phenotypes include metaboliser status categories which may relate to metabolism rates of active pharmaceutical ingredients. The metaboliser status categories are selected from any one or more of the group including ultra-rapid metabolisers, extensive metabolisers, intermediate metabolisers, and poor metabolisers.

The pharmacogenetic data 70 includes a plurality of drug reactions structurally linked to specific gene variations. The plurality of drug reactions includes specific pharmacological responses to active pharmaceutical ingredients.

The drug-drug interaction data 72 includes a plurality of known drug-drug contraindications structurally linked to specific active pharmaceutical ingredients. The drug-drug interaction data 72 includes metabolic pathway interference profiles. The metabolic pathway interference profiles may define pharmacokinetic interactions between the plurality of active pharmaceutical ingredients. The drug-drug interaction data 72 includes severity classifications. The severity classifications are indicative of a clinical risk level associated with the known contraindications. It is to be appreciated that the drug-drug interaction data 72 provides a generalised computational reference for identifying adverse polypharmacy effects independent of genetic factors.

The mapping database 34 includes the pharmacogenomic data 74. The pharmacogenomic data 74 includes a plurality of likely drug reactions structurally linked to active pharmaceutical ingredients and specific gene variations. In particular, the pharmacogenomic data 74 includes a plurality of biological pathways structurally linked to active pharmaceutical ingredients and specific gene variations. The plurality of biological pathways includes pharmacokinetic and pharmacodynamic pathways. The pharmacogenomic data 74 further includes therapeutic classifications structurally linked to the active pharmaceutical ingredients. The therapeutic classifications may define categorical groupings of the active pharmaceutical ingredients based on shared clinical purposes or common mechanisms of action. It is to be appreciated that the therapeutic classifications provide a structured computational reference enabling the system to extract suitable alternative therapies from within a shared categorical grouping when a specific active pharmaceutical ingredient is contraindicated. It is to be appreciated that the pharmacogenomic data 74 provides a relational data model for grouping the active pharmaceutical ingredients by shared biological pathways and therapeutic classes.

The mapping database 34 includes biological data 76. The biological data 76 includes any one or more of the group including disease-associated genetic markers, genotype-phenotype correlations, non-coding RNA analysis, gene function annotations, protein-protein interaction networks, pathway analysis results, regulatory elements, disease risk scores, polygenic risk scores, genetic traits or phenotypes, gene ontology annotations, genetic correlations, genome-wide association study (GWAS) results, and population-level genetic data.

The mapping database 34 includes pharmacological data 78. The pharmacological data 78 includes uses, effects and modes of action structurally linked to specific drugs. The pharmacological data 78 includes any one or more of the group including established drug-gene interaction pathways, adverse event probabilities, therapeutic efficacy thresholds, drug-herb interaction data, and toxicity risk factors.

The healthcare guidance data 32 includes clinical guidance data. The clinical guidance data includes a plurality of actionable clinical recommendations. The plurality of actionable clinical recommendations are structurally linked to the phenotypic data 68, pharmacogenetic data 70 and the drug-drug interaction data 72. Preferably, the plurality of actionable clinical recommendations are structurally linked to the phenotypic data 68, pharmacogenetic data 70, the drug-drug interaction data 72, the pharmacogenomic data 74, the biological data 76, and the pharmacological data 78. The plurality of actionable clinical recommendations are selected from any of the group including dosing recommendations, alternative treatment suggestions, risk stratification classifications, clinical monitoring protocols, cautionary clinical flags, and structured clinical action mappings. The structured clinical action mappings dictate specific actions the health practitioner 66 should take in response to any one or more of the group including a specific metabolic phenotype, a specific gene-drug interaction, a specific drug-drug interaction, a specific biological pathway taken by a specific pharmaceutical active ingredient, a specific biological data indication, and a specific pharmacological data indication. The alternative treatment suggestions are selected from any of the group including an alternative medication, an alternative therapeutic class, and an adjusted dosage regimen. The risk stratification classifications are selected from any of the group including toxicity risks, efficacy predictors, and metabolism rates.

The healthcare guidance data 32 includes age-specific rules. The age-specific rules are selected from any of the group including standard adult-based guidelines and paediatric-specific recommendations.

The healthcare guidance data 32 includes curated scientific source data. The curated scientific source data is selected from any of the group including pharmacogenomic consortia guidelines, regulatory labelling, and peer-reviewed literature. The curated scientific source data is configured to provide traceable reference links. It is to be appreciated that the traceable reference links ensure that the genetic-specific healthcare guidance is clinically validated and transparently verifiable by the health practitioner 66.

The mapping database 34 is configured as a relational database architecture. The data groups 35 are structurally linked via a plurality of associative data tables. The structural linking includes assigning a unique primary key to discrete data elements within each of the data groups 35. The structural linking may further include embedding a plurality of foreign keys across the associative data tables to computationally bind the data groups 35 together.

The data groups 35 are computationally categorised into database domains. The database domains may overlap with each other. The database domains include gene-related data 42, medication-related data 48, and healthcare-related data 54. The gene-related data 42 includes the phenotypic data 68, the biological data 76, the pharmacogenetic data 70, and the pharmacogenomic data 74. The medication-related data 48 includes the pharmacological data 78, the drug-drug interaction data 72, the pharmacogenetic data 70, and the pharmacogenomic data 74. The healthcare-related data 54 includes the healthcare guidance data 32. It is to be appreciated that assigning specific data groups, such as the pharmacogenetic data 70 and the pharmacogenomic data 74, to both the gene-related data 42 and the medication-related data 48 creates a computational overlap. The computational overlap establishes a relational bridge within the mapping database 34. The relational bridge enables the processor 12 to execute the multi-phase computational mapping operation 30, allowing a relational query to automatically traverse from the mapped gene-related data 44, through the overlapping data groups, to extract the corresponding mapped medication-related data 50 and the mapped healthcare-related data 56 without requiring manual human interpretation.

In an alternative embodiment, the mapping database 34 is configured as a directed graph database architecture. The data groups 35 are structurally linked via a plurality of computational nodes and directed edges. The structural linking includes storing discrete data elements within each of the data groups 35 as discrete computational nodes. The structural linking further includes computationally binding specific computational nodes together using the directed edges. Each directed edge includes a machine-readable relationship property, such as a biological pathway dependency, a metabolic rate interaction, or a drug-drug contraindication.

In the alternative embodiment, the data groups 35 are computationally categorised into database domains. The database domains overlap with each other. The database domains include gene-related data 42, medication-related data 48, and healthcare-related data 54. The gene-related data 42 includes the phenotypic data 68, the biological data 76, the pharmacogenetic data 70, and the pharmacogenomic data 74. The medication-related data 48 includes the pharmacological data 78, the drug-drug interaction data 72, the pharmacogenetic data 70, and the pharmacogenomic data 74. The healthcare-related data 54 includes the healthcare guidance data 32. It is to be appreciated that assigning specific data groups 35, such as the pharmacogenetic data 70 and the pharmacogenomic data 74, to both the gene-related data 42 and the medication-related data 48 creates a computational overlap. In the directed graph database architecture, the computational overlap is established by shared computational nodes and interconnecting directed edges. This computational overlap establishes a continuous associative path within the mapping database 34. The continuous associative path enables the processor 12 to execute the multi-phase computational mapping operation 30, allowing a graph traversal to automatically trace from the mapped gene-related data 44, along the directed edges through the overlapping data groups, to extract the corresponding mapped medication-related data 50 and the mapped healthcare-related data 56 without requiring manual human interpretation.

The mapping database 34 is in the form of a computational knowledge base. It is to be appreciated that configuring the mapping database 34 as a computational knowledge base ensures that the contents are structurally embedded with actionable logic for automated inference rather than passive data retrieval, thereby functioning as a structural component of a processing engine. The computational knowledge base includes a plurality of machine-readable logical relationships. The machine-readable logical relationships may structurally define interactions between the data groups 35 of the mapping database 34. The machine-readable logical relationships are executed via the foreign keys of the relational database architecture or the directed edges of the directed graph database architecture.

The computational knowledge base includes computable clinical logic. The computable clinical logic may provide machine-readable instructions which are executable to computationally govern the multi-phase mapping operation 30. It is to be appreciated that the computable clinical logic translates qualitative clinical guidelines into deterministic, machine-readable instructions, thereby enabling the processor 12 to automatically execute the multi-phase mapping operation 30 without requiring human clinical interpretation.

The machine-readable instructions include conditional algorithms. The conditional algorithms are configured to computationally govern dynamic evaluation of state-dependent variables. The state-dependent variables include dynamic clinical parameters derived from the user health condition information 28. It is to be appreciated that while the user genetic information 24 serves as a static biological baseline for the individual, the dynamic clinical parameters represent an ever-changing clinical snapshot, wherein a modification to the user health condition information 28 alters a computational state of the system 10 and necessitates algorithmic re-execution of the computable clinical logic to dynamically filter the mapped gene-specific healthcare information 38.

The mapping database 34 is securely hosted on any suitable computing infrastructure. The suitable computing infrastructure includes a distributed server architecture. Alternatively, or additionally, the mapping database 34 is hosted on any one or more of the group including a centralised server network, a cloud-based computing environment, a hybrid-cloud infrastructure, a peer-to-peer network, and an on-premise dedicated hardware system. It is to be appreciated that hosting the mapping database 34 on an on-premise dedicated hardware system may provide enhanced cryptographic security and compliance with localised data sovereignty regulations, whereas hosting on a cloud-based computing environment may provide dynamic scalability for the multi-phase computational mapping operation 30.

The system 10 includes a knowledge base updating operation. The knowledge base updating operation is configured to structurally integrate new data into the mapping database 34. The new data may correspond to contents of the mapping database 34. The knowledge base updating operation is configured to receive the new data which forms part of an external source. The external source is in the form of any one or more of the group including newly published pharmacogenomic guidelines, emerging clinical evidence, new clinical guidelines, emerging scientific evidence, and updated regulatory labelling. Structurally integrating the new data includes generating new discrete data elements and automatically assigning new primary keys and foreign keys, or generating new computational nodes and directed edges, within the mapping database 34. It is to be appreciated that integrating the new data alters the contents of the mapping database 34, thereby updating the machine-readable logical relationships and the computable clinical logic.

The multi-phase mapping operation 30 is initiated by the processor 12 automatically upon successful execution of the data storing operation. Alternatively, the mapping operation is initiated upon user selection of a selectable mapping element displayed via the user interface.

The multi-phase mapping operation 30 is executed in accordance with the computable clinical logic to generate individual-specific mapped data 80. The multi-phase mapping operation 30 is configured to store the individual-specific mapped data 80 against the individual profile 62.

The individual-specific mapped data 80 includes any one or more mapped data groups 82 including mapped phenotypic data 84, mapped pharmacogenetic data 86, mapped drug-drug interaction data 88, mapped pharmacogenomic data 90, mapped biological data 92, mapped pharmacological data 94, mapped risk data 96, mapped alternative treatment data 98, mapped healthcare data 56, and mapped condition-related healthcare data 100.

The mapped data groups 82 are computationally categorised into mapped database domains. The mapped database domains may overlap with each other. The mapped database domains include mapped gene-related data 44, mapped medication-related data 50, and mapped healthcare-related data 56. The mapped gene-related data 44 includes the mapped phenotypic data 84, the mapped biological data 92, the mapped pharmacogenetic data 86, and the mapped pharmacogenomic data 90. The mapped medication-related data 50 includes the mapped pharmacological data 94, the mapped drug-drug interaction data 88, the mapped pharmacogenetic data 86, and the mapped pharmacogenomic data 90. The mapped healthcare-related data 56 includes the mapped healthcare guidance data 56 and the mapped condition-related data 100.

It is to be appreciated that the individual-specific mapped data 80 defines a computationally filtered subset of the entire mapping database 34 which is filtered by the individual-specific user data 20. The computationally filtered subset includes any data within the mapping database 34 which is associated with the individual 22.

The individual-specific mapped data 80 further includes derived computational inferences. The derived computational inferences includes any one or more of the group including derived pharmacogenomic information, derived pharmacogenetic information, computationally established genotype-phenotype correlations, genetic traits or phenotypes, disease-associated genetic markers, genetic predispositions, carrier status for inherited conditions, disease risk scores, polygenic risk scores, pathway analysis results, ancestry or ethnic origins, and genetic relatedness to other individuals.

The individual-specific mapped data 80 forms the basis of the mapped gene-specific healthcare information 38 associated with the individual 22. The individual-specific mapped data 80 forms part of the output 36.

The multi-phase mapping operation 30 includes a plurality of phases for generating the individual-specific mapped data 80. Execution of the plurality of phases is initiated concurrently by the processor 12 upon initiation of the multi-phase mapping operation 30. The processor 12 is configured to manage computational dependencies between the plurality of phases during concurrent execution. Managing the computational dependencies includes temporarily suspending execution of a dependent phase pending generation of a required computational variable by an antecedent phase. Managing the computational dependencies further includes automatically resuming execution of the dependent phase upon receipt of the required computational variable. It is to be appreciated that this dependency-aware concurrent execution ensures optimal processing efficiency while maintaining chronological data integrity across the multi-phase mapping operation 30.

The plurality of phases includes the gene-mapping phase.

The gene-mapping phase includes a phenotypic mapping operation 102 for mapping the user genetic information 24 to the phenotypic data 68 stored on the mapping database 34. The phenotypic mapping operation 102 is executed by the processor 12. The phenotypic mapping operation 102 is configured to generate the mapped phenotypic data 84. The phenotypic mapping operation 102 is configured to computationally process and translate the user genetic information 24 into the mapped phenotypic data 84. The mapped phenotypic data 84 forms part of a phenotypic mapping output 104. The phenotypic mapping operation 102 includes querying the mapping database 34 using the user genetic information 24 to extract corresponding observable characteristics associated with the user genetic information 24. The phenotypic mapping operation 102 includes generating metabolic activity profiles based on a corresponding metabolic phenotype associated with the user genetic information 24. The metabolic activity profiles define a computational representation of how the individual 22 metabolises specific chemical compounds. The metabolic activity profiles form part of the phenotypic mapping output 104. The phenotypic mapping operation 102 includes storing the phenotypic mapping output 104 against the individual profile 22. It is to be appreciated that raw user genetic information 24, such as specific gene alleles, typically lacks direct clinical actionability until computationally translated into the individual-specific mapped phenotypic data 84, thereby establishing a functional and machine-readable biological baseline for the individual 22.

The gene-mapping phase includes a pharmacogenetic mapping operation 106 for mapping the user genetic information 24 to the pharmacogenetic data 74 stored on the mapping database 34. The pharmacogenetic mapping operation 106 is executed by the processor 12. The pharmacogenetic mapping operation 106 is configured to generate the mapped pharmacogenetic data 86. The mapped pharmacogenetic data 86 forms part of a pharmacogenetic mapping output 108.

The pharmacogenetic mapping operation 106 includes querying the mapping database 34 using the user genetic information 24 to extract corresponding drug reactions. The pharmacogenetic mapping operation 106 includes generating pharmacogenetic reaction profiles based on the corresponding drug reactions associated with the user genetic information 24. The pharmacogenetic reaction profiles define a computational representation of how the individual 22 reacts to specific pharmaceutical active ingredients. The mapped pharmacogenetic reaction profiles form part of the pharmacogenetic mapping output 108. The pharmacogenetic mapping operation 106 includes storing the pharmacogenetic mapping output 108 against the individual profile 62.

The gene-mapping phase includes a pharmacogenomic mapping operation 110 for mapping the user genetic information 24 to the pharmacogenomic data 74 stored on the mapping database 34. The pharmacogenomic mapping operation 110 is executed by the processor 12. The pharmacogenomic mapping operation 110 is configured to generate the mapped pharmacogenomic data 90. The mapped pharmacogenomic data 90 forms part of a pharmacogenomic mapping output 112. The pharmacogenomic mapping operation 110 includes querying the mapping database 34 using the user genetic information 24 to extract corresponding likely drug reactions. In particular, the pharmacogenomic mapping operation 110 includes querying the mapping database 34 using the user genetic information 24 to extract corresponding biological pathways and/or therapeutic classifications associated with active pharmaceutical ingredients and the user genetic information 24. The pharmacogenomic mapping operation includes generating mapped pharmacogenomic reaction profiles based on the corresponding biological pathways. The pharmacogenomic reaction profiles define a computational representation of how the individual 22 would likely react to specific pharmaceutical active ingredients. The mapped pharmacogenomic reaction profiles form part of the pharmacogenomic mapping output 112. The pharmacogenomic mapping operation 110 includes storing the pharmacogenomic mapping output 112 against the individual profile 62.

The plurality of phases includes the medication-mapping phase.

The medication-mapping phase includes a drug-drug interaction mapping operation 114 for mapping the user medication information 26 to the drug-drug interaction data 72 stored on the mapping database 34. The drug-drug interaction mapping operation 114 is executed by the processor 12. The drug-drug interaction mapping operation 114 is configured to generate the mapped drug-drug interaction data 88. The mapped drug-drug interaction data 88 forms part of a drug-drug interaction mapping output 116. The drug-drug interaction mapping operation 114 includes querying the mapping database 34 using the user medication information 26 to extract corresponding drug-drug contraindications. The drug-drug interaction mapping operation 114 includes generating drug-drug interaction profiles based on the corresponding drug-drug contraindications. The drug-drug interaction profiles define a computational representation of the specific drug-drug interactions taking place or having taken place historically, as a result of the user medication information 26 of the individual 22. The drug-drug interaction profiles form part of the drug-drug interaction mapping output 116. The drug-drug interaction mapping operation 114 includes storing the drug-drug interaction mapping output 116 against the individual profile 62.

The medication-mapping phase includes a pharmacological mapping operation 118 for mapping the user medication information 26 to the pharmacological data 78 stored on the mapping database 34. The pharmacological mapping operation 118 is executed by the processor 12. The pharmacological mapping operation 118 is configured to generate the mapped pharmacological data 94. The mapped pharmacological data 94 forms part of a pharmacological mapping output 120. The pharmacological mapping operation 118 includes querying the mapping database 34 using the user medication information 26 to extract corresponding uses, effects and modes of action of specific drugs. The pharmacological mapping operation 118 includes generating pharmacological profiles based on the corresponding uses, effects and modes of action of specific drugs. The pharmacological profiles define a computational representation of the uses, effects and modes of actions of specific drugs associated with the user medication information 26. The pharmacological profiles form part of the pharmacological mapping output 120. The pharmacological mapping operation 118 includes storing the pharmacological mapping output 120 against the individual profile 62.

The plurality of phases includes risk-mapping phase. The risk-mapping phase includes a risk mapping operation 124 for mapping the user medication information 26 and user genetic information 24 to risk-related information stored on the mapping database 34. The risk mapping operation 124 is executed by the processor 12. The risk mapping operation 124 is configured to generate the mapped risk data 96. The mapped risk data 96 forms part of a risk evaluation output 126. The risk mapping operation 124 includes a pharmacogenetic risk evaluation. The pharmacogenetic risk evaluation includes cross-referencing the user medication information 26 with the metabolic activity profiles generated by the phenotypic mapping operation 102. The pharmacogenetic risk evaluation is configured to identify a pharmacogenetic risk associated with the individual. The identified pharmacogenetic risk forms part of the mapped risk data 96. The risk evaluation operation 124 includes a drug-drug interaction risk evaluation. The drug-drug interaction risk evaluation includes cross-referencing the user medication information 26 against the drug-drug interaction data 72 contained within the mapping database 34. The drug-drug interaction risk evaluation is configured to identify a drug-drug interaction risk associated with the user medication information 26. The identified drug-drug interaction risk forms part of the mapped risk data 96. The risk evaluation operation 124 is configured to computationally map the identified risks to the mapped pharmacogenetic data 86 and mapped drug-drug interaction data 88 to generate the mapped risk data 96. The risk evaluation operation 124 includes storing the risk evaluation output 126 against the individual profile 62.

The risk mapping operation 124 is configured to execute the cross-referencing of the metabolic activity profiles generated by the phenotypic mapping operation 102 and the cross-referencing of the drug-drug interaction data 72 concurrently. It is to be appreciated that executing the pharmacogenetic risk evaluation and the drug-drug interaction risk evaluation concurrently enables the processor 12 to detect complex, compounding adverse reactions. This computational concurrency is believed to provide a distinct technical advantage by dynamically processing multi-variable clinical interactions that are mathematically prohibitive for a human practitioner to calculate manually.

The plurality of phases includes an alternative-treatment-mapping-phase. The alternative-treatment-mapping phase includes an alternative treatment mapping operation 130 for mapping the mapped risk data 96 generated by the risk mapping operation 124 to data contained in the mapping database 34 relating to alternative treatments. The alternative treatment mapping operation 130 is executed by the processor 12. The alternative treatment mapping operation 130 is initiated upon identification of the pharmacogenetic risk or the drug-drug interaction risk by the risk mapping operation 124. The alternative treatment mapping operation 130 is configured to computationally query the mapping database 34 to extract an alternative treatment suggestion associated with the identified risk. The alternative treatment mapping operation 130 is configured to generate the mapped alternative treatment data 98 based on the extracted alternative treatment suggestion. The mapped alternative treatment data 98 forms part of an alternative treatment output 132. The alternative treatment mapping operation 130 includes storing the alternative treatment output 132 against the individual profile 62. It is to be appreciated that extracting the alternative treatment suggestion computationally bridges the gap between risk identification and clinical resolution. The alternative treatment extraction is believed to ensure the system functions as a proactive, automated guidance engine rather than a passive alert mechanism, dynamically presenting the health practitioner 66 with safe, computationally verified treatment pathways.

The plurality of phases includes a biological-mapping phase. The biological mapping phase includes a biological mapping operation 136 for mapping the user genetic information 24, the user medication information 26 and the user health condition information 28 to the biological data 76 stored on the mapping database 34. The biological mapping operation 136 is executed by the processor 12. The biological mapping operation 136 is configured to generate the mapped biological data 92. The mapped biological data 92 forms part of a biological mapping output 138. The biological mapping operation 136 includes querying the mapping database 34 using the user genetic information 24, the user medication information 26 and the user health condition information 28 to extract corresponding biological data. The biological mapping operation 136 includes generating biological reaction profiles based on the corresponding biological data. The biological reaction profiles define a computational representation of biological reactions experienced and/or likely to be experienced by the individual 22 based on the user genetic information 24, the user medication information 26 and the user health condition information 28. The biological reaction profiles form part of the biological mapping output 138. The biological mapping operation 136 includes storing the biological mapping output 138 against the individual profile 62.

The plurality of phases includes the healthcare-mapping phase. The healthcare-mapping phase includes a healthcare mapping operation 140 for mapping any one or more system operation outputs to the healthcare guidance data 32 stored on the mapping database 34.

The system operation outputs include the phenotypic mapping output, the pharmacogenetic mapping output, the drug-drug interaction mapping output, the pharmacogenomic mapping output, the biological mapping output, the pharmacological mapping output, the risk evaluation output, and the alternative treatment output.

The healthcare mapping operation 140 is executed by the processor 12. The healthcare mapping operation 140 is configured to generate the mapped healthcare data 56. The mapped healthcare data 56 forms part of a healthcare mapping output 142.

The healthcare mapping operation 140 includes querying the mapping database 34 using any one or more of the system operation outputs to extract corresponding actionable clinical recommendations. The healthcare mapping operation 140 includes generating a healthcare guidance profile based on the corresponding actionable clinical recommendations. The healthcare guidance profile defines a computational representation of the specific actionable clinical recommendations for the individual 22 based on any one or more of the system operational outputs. The healthcare guidance profile forms part of the healthcare guidance mapping output 142. The healthcare guidance profile forms part of the mapped gene-specific healthcare guidance information 38. The healthcare guidance mapping operation 140 includes storing the healthcare mapping output 142 against the individual profile 62.

The plurality of phases includes a health-condition-mapping phase. The health-condition-mapping phase includes a condition mapping operation 146 for mapping the user health condition information 28 to the individual-specific mapped data 80. In particular, the condition mapping operation 146 is configured to map the user health condition information 28 to the mapped gene-related data 44, the mapped medication-related data 50, and the mapped healthcare-related data 56 to generate the mapped condition-related healthcare data 100 associated with the individual 22 to be included in the output 36. It is to be appreciated that the mapped condition-related healthcare data 100 pertains to a specific health condition of the individual 22 and that the mapped condition-related healthcare data 100 can be used to filter all the individual-specific mapped data 80 to easily obtain the mapped data most relevant to the specific health condition.

The system 10 includes an output classification operation. The output classification operation is executed by the processor 12. The output classification operation is configured to computationally process and categorise any one or more of the system operation outputs into a plurality of classification categories. The plurality of classification categories is indicative of varying levels of clinical risk. The plurality of classification categories is selected from any of the group including standard administration, therapeutic caution, and alternative treatment recommended. The output classification operation is configured to assign a distinct visual identifier to each of the classification categories. The distinct visual identifier includes a colour-coded classification. The output classification operation is configured to compile the categorised system operation outputs and the assigned visual identifiers to generate a classification profile. The classification profile forms part of the mapped gene-specific healthcare guidance 38. The output classification operation includes storing the classification profile against the individual profile 62. It is to be appreciated that the output classification operation computationally structures the complex biological and pharmacological data into a visually discernible hierarchy. This structural categorisation is believed to minimise the cognitive load on the health practitioner 66, facilitating rapid, computationally assisted clinical decision-making without requiring the practitioner 66 to manually interpret the underlying raw data.

The system 10 includes a dynamic re-mapping operation 148. The dynamic re-mapping operation 148 is configured to automatically re-execute the multi-phase mapping operation 30. The dynamic re-mapping operation 148 is initiated upon detection of a system variable update. The system variable update is classified as either a local state modification or a global state modification. The local state modification includes a modification to the individual-specific user data 20, preferably the user medication information 26 or the user health condition information 28. The global state modification includes an update or modification to the contents of the mapping database 34, preferably after execution of the knowledge base updating operation. It is to be appreciated that the dynamic re-mapping operation 148 ensures that the individual-specific mapped data 80 remains continuously accurate, automatically generating updated healthcare guidance information without requiring manual practitioner initiation whenever the individual 22 changes medications, updates any user health condition information 28 or whenever new data is integrated into the system.

The dynamic re-mapping operation 148 is configured to computationally filter a list of active pharmaceutical ingredients according to the current user medication information 26 or the current user health condition information 28. It is to be appreciated that because the current user medication information 26 or the current user health condition information 28 is updated dynamically by the health practitioner 66 during a clinical consultation, the processor 12 executes the dynamic re-mapping operation 148 to output a dynamically filtered, real-time clinical assessment based on the most recent health status of the individual 22.

The system includes an authorisation operation 150 for enabling the individual 22 to authorise the health practitioner 66 to access the individual profile 62, thereby assisting the health practitioner 66 to provide the mapped gene-specific healthcare guidance information 38 to the individual 22. The authorisation operation 150 is executed by the processor 12 to securely manage access to the repository 60. The authorisation operation 150 includes displaying a login visual to a user via the user interface. The user includes the individual 22 or a health practitioner 66. The login visual includes data input fields configured to receive the login details of the user. The authorisation operation 150 includes a multi-factor authentication module. The multi-factor authentication module is configured to receive a one-time pin (OTP) or a time-based one-time pin (TOTP) to successfully authenticate the user.

The processor 12 is configured to execute the authorisation operation 150 via a plurality of access interfaces. The plurality of access interfaces include a dedicated client application executable on a personal computing device associated with the user. Alternatively, the plurality of access interfaces includes a web-based application accessible via an internet browser.

The authorisation operation 150 includes a practitioner-initiated access protocol. The practitioner-initiated access protocol is initiated by a registered health practitioner 66 submitting an access request associated with a specific registered individual. The processor 12 is configured to transmit the access request to a personal computing device associated with the individual 22. The access request includes identification information of the requesting health practitioner 66 and a computational prompt configured to receive an access grant or an access denial from the individual 22. Upon receiving the access grant, the processor 12 is configured to generate an access token, such as a TOTP, for the health practitioner 66 to input via the user interface. The practitioner-initiated access protocol is configured to computationally restrict access to the individual's profile 62 to a predefined time limit.

The authorisation operation 150 includes an individual-initiated access protocol. The individual-initiated access protocol is configured to display a directory of registered health practitioners to the individual 22. The individual-initiated access protocol is configured to receive a selection of a specific registered health practitioner 66 from the directory. Upon receiving the selection, an access notification including a secure access link is generated and transmitted to a computing device associated with the selected health practitioner 66.

The authorisation operation 150 includes an administrative access protocol. The administrative access protocol is configured to permit a system service provider to securely access the repository 60. The administrative access protocol enables the system service provider to computationally alter or update system variables to maintain continuous system operability.

The processor 12 is configured to execute a plurality of integrated computational modules. The integrated computational modules are initiatable by the health practitioner 66. The integrated computational modules include any one or more of the group including a query module, a dual-view rendering engine, a longitudinal medication history module, and a comprehensive variant access module.

The query module is configured to receive a manual query input from the health practitioner 66 via the user interface. The manual query input includes any one or more of the group including a proposed active pharmaceutical ingredient, a therapeutic classification, and an identified clinical indication. The query module is configured to computationally execute a simulated evaluation of the manual query input against the individual-specific mapped data prior to prescription. The simulated evaluation includes dynamically filtering contents of the mapping database 34 based on user genetic information 24, specific user medication information 26, and specific user health condition information 28 of the individual 22. It is to be appreciated that the query module enables predictive scenario testing, allowing the health practitioner 66 to computationally verify the safety of a proposed treatment or instantly generate a list of genotype-compatible alternatives within a specific therapeutic class without permanently modifying the core user medication data.

The dual-view rendering engine is configured to structurally format a graphical user interface. The dual-view rendering engine is configured to computationally toggle the graphical user interface between a clinical summary interface and an evidence-based interface. The clinical summary interface is configured to display mapped structured clinical action mappings and mapped distinct visual identifiers obtained from the output 36 of the multi-phase mapping operation 30. The evidence-based interface is configured to display mapped scientific source data and mapped traceable reference links obtained from the output 36 of the multi-phase mapping operation 30. It is to be appreciated that the dual-view rendering engine dynamically manages the cognitive load on the health practitioner 66 by separating rapid-decision data from deep-dive scientific verification data, thereby improving visual interpretability while preserving the underlying integrity of system-generated outputs.

The longitudinal medication history module is configured to chronologically aggregate the historical user medication information. The longitudinal medication history module may retrospectively execute the risk mapping operation on the historical user medication information using the individual-specific mapped phenotypic data 84. It is to be appreciated that retrospectively mapping past medications against the newly translated phenotypic baseline allows the processor 12 to computationally identify root causes of historical adverse drug reactions or prior treatment failures.

The comprehensive variant access module is configured to provide structured access to raw user genetic information. The comprehensive variant access module is configured to display specific gene alleles and genotypic markers independent of the individual-specific mapped phenotypic data 84. The raw user genetic information includes information regarding genetic variants beyond those currently implicated in active clinical recommendations. It is to be appreciated that the comprehensive variant access module accommodates deep-dive specialist review, providing the health practitioner 66 with transparent access to underlying genetic architecture of the individual 22.

It is to be appreciated that while the multi-phase mapping operation 30 functions as an automated processing pipeline, the integrated computational modules provide the health practitioner 66 with specific, on-demand digital tools to manually interrogate data and visually customise the gene-specific healthcare guidance.

The system 10 includes a reporting operation 152 executable by the processor 12. The reporting operation 152 is configured to computationally compile the individual-specific mapped data 80 and the gene-specific healthcare guidance data 38 into a secure digital document, such as a portable document format (PDF) report.

The reporting operation 152 includes a summary reporting operation. The summary reporting operation is configured to generate a summary report. The summary report is structurally restricted to the current user health condition information 28 and/or the current user medication information 26 of the individual 22. The summary reporting operation is configured to filter the individual-specific mapped data 80 to display pharmacogenetic insights, identified pharmacogenetic risks, and specific alternative treatment suggestions solely associated with a current medication regimen of the individual 22. It is to be appreciated that the summary report provides a targeted, highly actionable snapshot optimised for immediate clinical decision-making regarding the active pharmaceutical ingredients currently prescribed to the individual 22.

The reporting operation 152 includes a comprehensive reporting operation. The comprehensive reporting operation is configured to generate a comprehensive report. The comprehensive report includes the complete output 36 of the multi-phase mapping operation 30, and an output of any further system operations. Alternatively, the comprehensive report includes a complete computational mapping across a comprehensive list of active pharmaceutical ingredients. The comprehensive report may detail all potential gene-medicine interactions, pharmacogenomic pathways, and risk stratification classifications associated with the foundational genotypic data of the individual 22, independent of the current medication regimen.

The summary report and the comprehensive report include the distinct visual identifiers, cautionary clinical flags, and structured clinical action mappings to rapidly convey therapeutic efficacy and potential risks. The reporting operation 152 is configured to embed a clinical audit trail within the reports. The clinical audit trail includes the traceable reference links and the curated scientific source data. It is to be appreciated that embedding the clinical audit trail allows the health practitioner 66 to transparently view and verify the structured, rule-based clinical logic utilised by the processor 12 to reach a particular recommendation or alternative treatment suggestion.

The reporting operation 152 is dynamically responsive to the dynamic re-mapping operation 148. The processor 12 is configured to automatically regenerate the summary report and the comprehensive report upon detection of a system variable update. It is to be appreciated that the dynamic regeneration ensures that any exported digital document remains continuously aligned with the most recent health status of the individual 22 and latest global clinical guidelines.

The system includes a notification operation 154 executable by the processor 12. The notification operation 154 is computationally linked to the dynamic re-mapping operation 148. The notification operation 154 is initiated upon execution of the dynamic re-mapping operation 148 resulting specifically from a global state modification. The notification operation 154 includes computationally comparing newly generated individual-specific mapped data against historically stored individual-specific mapped data. The notification operation 154 includes computationally detecting a clinical deviation between the newly generated individual-specific mapped data and the historically stored data. The clinical deviation indicates that the newly integrated scientific source data alters or influences a previously generated actionable clinical recommendation. Upon detecting the clinical deviation, the notification operation is configured to dynamically generate a targeted clinical alert. The notification operation is configured to automatically transmit the targeted clinical alert to the individual 22 or the authorised health practitioner 66 via the plurality of access interfaces or personal computing devices associated with the individual 22 or health practitioner 66. It is to be appreciated that the notification operation 154 transforms the system 10 from a passive reference tool into a proactive, state-aware clinical surveillance engine, ensuring that all historical patient mappings are continuously and automatically audited against the latest scientific literature without requiring manual practitioner initiation.

1. A genetic healthcare guidance system which includes:
a processor with access to a memory containing instructions executable by the processor to perform operations including:
a data acquisition operation for acquiring individual-specific user data from an individual, the individual-specific user data including user genetic information, user medication information, and user health condition information; and
a multi-phase computational mapping operation for mapping the individual-specific user data to healthcare guidance data contained in a mapping database to generate an output including mapped gene-specific healthcare guidance information associated with the individual, the multi-phase computational mapping operation including:
a gene-mapping phase for mapping the user genetic information to gene-related data contained in the mapping database to generate mapped gene-related data associated with the individual;
a medication-mapping phase for mapping the user medication information to medication-related data contained in the mapping database to generate mapped medication-related data associated with the individual; and
a healthcare-mapping phase for mapping the mapped gene-related data and the mapped medication-related data to healthcare-related data contained in the mapping database to generate mapped healthcare data associated with the individual to be included in the output.

2. The genetic healthcare guidance system as claimed in claim 1 wherein the mapping database includes any data groups including phenotypic data, pharmacogenetic data, drug-drug interaction data, pharmacogenomic data, biological data, pharmacological data, and healthcare guidance data.

3. The genetic healthcare guidance system as claimed in claim 2 wherein the healthcare guidance data includes clinical guidance data.

4. The genetic healthcare guidance system as claimed in claim 3 wherein the clinical guidance data includes a plurality of actionable clinical recommendations.

5. The genetic healthcare guidance system as claimed in claim 4 wherein the plurality of actionable clinical recommendations are structurally linked to the phenotypic data, pharmacogenetic data, the drug-drug interaction data, the pharmacogenomic data, the biological data, and the pharmacological data.

6. The genetic healthcare guidance system as claimed in claim 4 wherein the plurality of actionable clinical recommendations are selected from any of the group including dosing recommendations, alternative treatment suggestions, risk stratification classifications, clinical monitoring protocols, cautionary clinical flags, and structured clinical action mappings.

7. The genetic healthcare guidance system as claimed in claim 1 wherein the healthcare guidance data includes curated scientific source data.

8. The genetic healthcare guidance system as claimed in claim 1 wherein the mapping database is configured as a relational database architecture.

9. The genetic healthcare guidance system as claimed in claim 2 wherein the mapping database is in the form of a computational knowledge base.

10. The genetic healthcare guidance system as claimed in claim 9 wherein the computational knowledge base includes a plurality of machine-readable logical relationships which structurally define interactions between the data groups of the mapping database.

11. The genetic healthcare guidance system as claimed in claim 1 wherein the multi-phase mapping operation is executed in accordance with computable clinical logic to generate individual-specific mapped data.

12. The genetic healthcare guidance system as claimed in claim 11 wherein the individual-specific mapped data includes any mapped data groups including mapped phenotypic data, mapped pharmacogenetic data, mapped drug-drug interaction data, mapped pharmacogenomic data, mapped biological data, mapped pharmacological data, mapped risk data, mapped alternative treatment data, mapped healthcare data, and mapped condition-related healthcare data.

13. The genetic healthcare guidance system as claimed in claim 12 wherein the individual-specific mapped data forms the basis of the gene-specific healthcare information associated with the user.

14. The genetic healthcare guidance system as claimed in claim 1 wherein the multi-phase mapping operation includes a plurality of phases for generating the individual-specific mapped data.

15. The genetic healthcare guidance system as claimed in claim 14 wherein execution of the plurality of phases are initiated concurrently by the processor upon initiation of the multi-phase mapping operation.

16. The genetic healthcare guidance system as claimed in claim 15 wherein the processor is configured to manage computational dependencies between the plurality of phases during concurrent execution.

17. The genetic healthcare guidance system as claimed in claim 16 wherein managing the computational dependencies include temporarily suspending execution of a dependent phase pending generation of a required computational variable by an antecedent phase and automatically resuming execution of the dependent phase upon receipt of the required computational variable.

18. The genetic healthcare guidance system as claimed in claim 1 which includes a dynamic re-mapping operation configured to automatically re-execute the multi-phase mapping operation upon detection of a system variable update.

19. The genetic healthcare guidance system as claimed in claim 18 wherein the system variable update is classified as either a local state modification or a global state modification, wherein the local state modification includes a modification to the individual-specific user data, and wherein the global state modification may include an update or modification to the contents of the mapping database.

20. The genetic healthcare guidance system as claimed in claim 1 which includes include an authorisation operation for enabling an individual to authorise a health practitioner to access an individual profile, thereby assisting the health practitioner to provide the gene-specific healthcare guidance information to the individual.
